SEQUENCE LISTING (2 sequences)

SEQ ID NO: 1 - protein, 10 aa, Artificial Sequence
  Description of Artificial Sequence Synthetic peptide
EQKLISEEDL

SEQ ID NO: 2 - protein, 8 aa, Artificial Sequence
  Description of Artificial Sequence Synthetic peptide
DYKDDDDK

(19) United States
(12) Patent Application Publication
(10) Pub. No.: US 2016/0106799 A1
(43) Pub. Date: Apr. 21, 2016

(54) Carbohydrate Compounds for Nutritional and Therapeutic Use

(71) Applicant: SIAMAB THERAPEUTICS, INC., Newton, MA (US)

(72) Inventors: Mai Zhang, Carlsbad, CA (US); Darius Ghaderi, Laupheim (DE); Jeffrey Behrens, Newton, MA (US); Julie DeSander, Arlington, MA (US); Ajit Varki, La Jolla, CA (US)

(73) Assignee: SIAMAB THERAPEUTICS, INC., Newton, MA (US)

(21) Appl. No.: 14/893,971

(22) PCT Filed: May 29, 2014
(86) PCT No.: PCT/US14/39963

§ 371 Date: Nov. 25, 2015

Related U.S. Application Data
(60) Provisional application No. 61/828,334, filed on May 29, 2013.
(60) Provisional application No. 61/833,972, filed on Jun. 12, 2013.
(60) Provisional application No. 61/840,519, filed on Jun. 28, 2013.
(60) Provisional application No. 61/864,759, filed on Aug. 12, 2013.
(60) Provisional application No. 61/941,522, filed on Feb. 19, 2014.

(51) Int. Cl. A61K 38/01; G01N 33/68

(52) U.S. Cl. CPC A61K 38/018; G01N 33/6863; A23L 1/3053; A23L 1/293; G01N 2333/57; G01N 2333/5409; G01N 2333/5412; G01N 2333/525; G01N 2333/523; G01N 2800/52; G01N 2800/7095; A23V 2002/00

(57) ABSTRACT

Embodiments of the present invention provide compositions and methods for the modulation of sialic acid levels. In some embodiments, compositions disclosed herein comprise nutraceuticals as functional agents.

### CROSS REFERENCE TO RELATED APPLICATIONS

This application claims priority to U.S. Provisional Patent Application No. 61/828,334, filed May 29, 2013, entitled Carbohydrate Compounds for Nutritional and Therapeutic Use, U.S. Provisional Patent Application No. 61/840,519, filed Jun. 28, 2013, entitled Carbohydrate Compounds for Nutritional and Therapeutic Use, U.S. Provisional Patent Application No. 61/833,972, filed Jun. 12, 2013, entitled Carbohydrate Compounds for Nutritional and Therapeutic Use, U.S. Provisional Patent Application No. 61/864,759 filed Aug. 12, 2013, entitled Carbohydrate Compounds for Nutritional and Therapeutic Use and U.S. Provisional Patent Application No. 61/941,522 filed Feb. 19, 2014, entitled Carbohydrate Compounds for Nutritional and Therapeutic Use, the contents of each of which are herein incorporated by reference in their entirety.

### FIELD OF THE INVENTION

This invention relates to compounds and compositions, including, but not limited to carbohydrates for the maintenance and/or promotion of general health and well-being as well as for the treatment of diseases, disorders and/or conditions.

### BACKGROUND OF THE INVENTION

Sialic acids are amino sugars that are essential components of glycoconjugates found in nearly all biological organisms and viral particles. In humans sialic acids are components of glycoconjugate proteins and lipids found in many tissues of the body, especially the brain.

N-acetylneuraminic acid (Neu5Ac) and N-glycolylneuraminic acid (Neu5Gc) are the two major sialic acids on mammalian cell surfaces. Neu5Ac and Neu5Gc differ only in that Neu5Gc comprises an additional oxygen atom associated with the chemical group attached to carbon 5. Due to the loss of a functional gene, humans can only synthesize sialic acid in the form of Neu5Ac, but not Neu5Gc. However Neu5Gc can be metabolically incorporated into humans from animal-derived dietary sources such as red meats (Tangvoranuntakul, P. et al., Human uptake and incorporation of an immunogenic nonhuman dietary sialic acid. Proc Natl Acad Sci USA. 2003 Oct. 14; 100(21):12045-50; Nguyen, D. H. et al., Effects of natural human antibodies against a nonhuman sialic acid that metabolically incorporates into activated and malignant immune cells. J Immunol. 2005 Jul. 1; 175(1):228-36; U.S. Pat. No. 7,682,794, U.S. Pat. No. 8,084,219, US2010/0293624, US2011/0195921, US2012/0142903, WO2010030666 and WO2006133356, the contents of each of which are herein incorporated by reference in their entirety). Neu5Gc is significantly abundant among human tumors (Higashi, H. et al., Characterization of N-glycolylneuraminic acid-containing gangliosides as tumor-associated Hanganutziu-Deicher antigen in human colon cancer. Cancer Res. 1985 August; 45(8):3796-802; Miyoshi I. et al., Detection of 4-O-acetyl-N-glycolylneuraminyl lactosylceramide as one of tumor-associated antigens in human colon cancer tissues by specific antibody. Mol Immunol. 1986. 23: 631-638; Hirabayashi, Y. et al., Occurrence of tumor-associated ganglioside antigens with Hanganutziu-Deicher antigenic activity on human melanomas. Jpn J Cancer Res. 1987. 78: 614-620; Kawachi. S, et al., Heterophile Hanganutziu-Deicher antigen in ganglioside fractions of human melanoma tissues. Int Arch Allergy Appl Immunol. 1988. 85: 381-383; Devine, P. L. et al., The breast tumor-associated epitope defined by monoclonal antibody 3E1.2 is an O-linked mucin carbohydrate containing N-glycolylneuraminic acid. Cancer Res. 1991. 51: 5826-5836; Malykh, Y. N. et al, N-Glycolylneuraminic acid in human tumours. Biochimie. 2001. 83: 623-634) and remarkably low in normal human tissues, which had been overlooked for several decades (Diaz, S. L. et al., Sensitive and specific detection of the non-human sialic Acid N-glycolylneuraminic acid in human tissues and biotherapeutic products. PLoS One. 2009. 4: e4241; Tangvoranuntakul, P. et al., Human uptake and incorporation of an immunogenic nonhuman dietary sialic acid. Proc Natl Acad Sci USA. 2003. 100: 12045-12050; Varki, A. et al., Multiple changes in sialic acid biology during human evolution. Glycoconj J. 2009. 26: 231-245). Interestingly, the metabolic accumulation of diet-derived Neu5Gc in cancer tissue is elevated compared to healthy human tissues. This is likely explained by at least three factors: rapid growth with underproduction of competing endogenous Neu5Ac, enhanced macropinocytosis induced by growth factors (Dharmawardhane, S. et al., Regulation of macropinocytosis by p21-activated kinase-1. Mol Biol Cell. 2000 October; 11(10):3341-52; Simonsen, A. et al., The role of phosphoinositides in membrane transport. Curr Opin Cell Biol. 2001 August; 13(4):485-92; Johannes, L. et al., Clathrin-dependent or not: is it still the question? Traffic. 2002 July; 3(7):443-51; Amyere, M. et al., Origin, originality, functions, subversions and molecular signaling of macropinocytosis. Int J Med Microbiol. 2002 February; 291(6-7):487-94), and the upregulation of gene expression of the lysosomal sialic acid transporter gene sialin by hypoxia (Yin, J. et al., Hypoxic culture induces expression of sialin, a sialic acid transporter, and cancer-associated gangliosides containing non-human sialic acid on human cancer cells. Cancer Res. 2006 Mar. 15; 66(6):2937-45). In addition, all humans tested to date comprise a polyclonal antibody reservoir against non-human Neu5Gc, which makes it the first example of a xeno-autoantigen (Padler-Karavani, V. et al., Diversity in specificity, abundance, and composition of anti-Neu5Gc antibodies in normal humans: potential implications for disease. Glycobiology. 2008 October; 18(10):818-30; Varki, N. M. et al., Biomedical differences between human and nonhuman hominids: potential roles for uniquely human aspects of sialic acid biology. Annu Rev Pathol. 2011; 6:365-93).

Current evidence suggests that the accumulation of Neu5Gc in humans, may be harmful. The accumulation of dietary Neu5Gc in malignant tumors in the face of an anti-Neu5Gc response was shown to facilitate tumor progression by inducing a low-grade chronic inflammation (Hedlund, M. et al., Evidence for a human-specific mechanism for diet and antibody-mediated inflammation in carcinoma progression. Proc Natl Acad Sci USA. 2008 Dec. 2; 105(48):18936-41). Thus, compositions and methods for the reduction of Neu5Gc levels in subjects are needed.

### SUMMARY OF THE INVENTION

In some embodiments, the present invention provides nutraceutical compositions comprising one or more functional agents comprising N-acetylneuraminic acid (Neu5Ac), and one or more excipient. In some embodiments, such Neu5Ac is comprised in one or more glycans. In some embodiments, functional agents are comprised in one or more functional foods. In some embodiments, functional foods may be selected from the group consisting of edible birds nest and avian egg whites. In some embodiments, functional agents comprise one or more glycoproteins. In some embodiments, glycoproteins comprise glycomacropeptide (GMP). In some embodiments, GMP may be hypersialylated. In some embodiments, functional foods comprise one or more medical foods. In some embodiments, nutraceutical compositions of the present invention may comprise one or more anti-inflammatory agents. In some embodiments, nutraceutical compositions of the present invention may comprise one or more lipid vehicles. In some embodiments, nutraceutical compositions are provided wherein one or more functional agents are applied to one or more functional foods by a method selected from the group consisting of sprinkling, mixing and coating.

In some embodiments, the present invention provides methods for reducing or eliminating N-glycolylneuraminic acid (Neu5Gc) levels in a subject by providing a nutraceutical composition. In some embodiments, such nutraceutical compositions are formulated for oral administration. In some embodiments, the present invention provides methods of evaluating the ability of a nutraceutical composition to reduce or eliminate Neu5Gc levels in a subject comprising the steps of: administering nutraceutical compositions to a subject, obtaining a sample from the subject and determining the level of Neu5Gc in the sample. In some embodiments, such methods further comprise determining the level of anti-Neu5Gc antibody levels in such subject. In some embodiments, the present invention provides methods of reducing or eliminating Neu5Gc from one or more tissues in a subject by providing a nutraceutical composition, wherein tissues are comprised in an organ selected from the group consisting of intestine, heart and aorta. In some embodiments, methods for preventing, reducing or eliminating inflammation in a subject are provided comprising providing a nutraceutical composition. In some embodiments, methods of treating a subject with Phenylketonuria are provided comprising administering a nutraceutical composition of the present invention.

In some embodiments, the present invention provides a method of evaluating the ability of a nutraceutical composition to modulate the level of one or more inflammatory biomarkers in a subject comprising the steps of administering a nutraceutical composition of the present invention to a subject, obtaining a first sample from the subject, obtaining a second sample from the subject from about 1 week to about 12 weeks after obtaining the first sample, determining the level of one or more inflammatory biomarkers in the first sample and the level of one or more inflammatory biomarkers in the second sample, and comparing the level of the inflammatory biomarkers in the second sample to the level of the inflammatory biomarkers in the first sample.

In another embodiment, the present invention provides a method of incorporating sialic acid into one or more tissues of a subject comprising administering to the subject a nutraceutical composition according to the present invention. In such methods, the tissue may comprise tracheal, lung and/or skin tissue. Other aspects provide methods of increasing the level of sialic acid in one or more tissues of a subject comprising administering to the subject a nutraceutical composition according to any of those described herein. According to some such methods, the tissue may comprise tracheal, lung and/or skin tissue.

In some embodiments, the present invention provides methods of preparing processed meat products by combining a meat with one or more functional agents. In some cases, such functional agents include sialic acid. In some cases, the functional agents have a total sialic acid content equal to the total amount of Neu5Ac plus the total amount of Neu5Gc. The total sialic acid content may, in some cases, be made up only of Neu5Ac. In other cases, the total sialic acid content is made up of more than 50% Neu5Ac.

In some embodiments, methods of preparing processed meat products may include the combination of a meat with glycomacropeptide (GMP.) According to such methods, the meat may be from cows, pigs, goats, sheep, chickens or fish. In some cases, such methods may include curing, heating, cooking, drying and/or fermenting the meat. In some cases, the meat may be finely textured meat. Meat products produced may include patties, nuggets, sausages or loaves.

### BRIEF DESCRIPTION OF THE FIGURES

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 depicts the results of immunohistochemical analysis of tracheal tissue sections from mice involved in the feeding study described in Example 3.

FIG. 2 depicts the results of immunohistochemical analysis of periodate treated tracheal tissue sections from mice involved in the feeding study described in Example 3.

FIG. 3 depicts the results of immunohistochemical analysis of lung tissue sections from mice involved in the feeding study described in Example 3. Results from Group 1 and 2 mice are shown in FIG. 3A and results from Group 3 and 4 mice are shown in FIG. 3B.

FIG. 4 depicts the results of immunohistochemical analysis of skin tissue sections from mice involved in the feeding study described in Example 3. Results from Group 1 and 2 mice are shown in FIG. 4A and results from Group 3 and 4 mice are shown in FIG. 4B.

### DETAILED DESCRIPTION

In some embodiments, the present invention provides compounds and/or compositions for modulating sialic acid levels and profiles in subjects. In some embodiments, such compounds and/or compositions comprise sialic acid. In some embodiments, such sialic acids are administered free and/or as part of a glycan and/or glycoconjugate (including, but not limited to glycoproteins). As used herein, the term “sialic acid” refers to any one or more members from a family of sialic acids comprising N- and O-substituted derivatives of neuraminic acid. Neuraminic acid (Neu) is a monosaccharide comprising a 9-carbon backbone. The three most common forms of sialic acid are N-glycolylneuraminic acid (Neu5Gc), N-acetylneuraminic acid (Neu5Ac) and 2-keto-3-deoxynonic acid (Kdn) with Neu5Ac and Neu5Gc dominating in mammals with the exception of humans where Neu5Ac is the dominant form.

Sialic acids are typically incorporated onto glycan chains as the last or “capping” residue added to the non-reducing end of the chain. Such chains are usually present at the cell surface or secreted from cells and/or organisms. At the ends of such chains, sialic acids mediate many interactions between cells as well as between cells and their environment. Influencing such interactions, core structures of sialic acids may be catalytically modified by one or more enzymes, including, but not limited to cytodine monophosphate (CMP)N-acetylneuraminic acid hydroxylase which is capable of converting Neu5Ac to Neu5Gc (Shaw, L. et al., The biosynthesis of N-glycoloylneuraminic acid occurs by hydroxylation of the CMP-glycoside of N-acetylneuraminic acid. Biol Chem Hoppe Seyler. 1988 June; 369(6):477-86; Shaw, L. et al., CMP-N-acetylneuraminic acid hydroxylase from mouse liver and pig submandibular glands. Interaction with membrane-bound and soluble cytochrome b5-dependent electron transport chains. Eur J Biochem. 1994 Feb. 1; 219(3):1001-11; Takematsu, H. et al., Reaction mechanism underlying CMP-N-acetylneuraminic acid hydroxylation in mouse liver: formation of a ternary complex of cytochrome b5, CMP-N-acetylneuraminic acid, and a hydroxylation enzyme. J Biochem. 1994 March; 115(3):381-6; Kawano, T. et al., Molecular cloning of cytidine monophospho-N-acetylneuraminic acid hydroxylase. Regulation of species- and tissue-specific expression of N-glycolylneuraminic acid. J Biol Chem. 1995 Jul. 7; 270(27):16458-63; Kelm, S. et al., Sialic acids in molecular and cellular interactions. Int Rev Cytol. 1997; 175:137-240; Angata, T. et al., Chemical diversity in the sialic acids and related alpha-keto acids: an evolutionary perspective. Chem Rev. 2002 February; 102(2):439-69; Varki, A., Colloquium paper: uniquely human evolution of sialic acid genetics and biology. Proc Natl Acad Sci USA. 2010 May 11; 107 Suppl 2:8939-46).

Harmful disorders and conditions may be associated with Neu5Gc. In some embodiments, many health and performance benefits may be attributed to the administration of Neu5Ac. Ingestion of Neu5Ac may compete metabolically for glycoconjugate incorporation with Neu5Gc, making Neu5Ac administration a therapeutic for cancer, inflammation and/or other conditions associated with Neu5Gc in subjects. Additionally, sialic acid is important for mammalian brain development where it has been shown to improve learning and memory (Wang, B. et al., Dietary sialic acid supplementation improves learning and memory in piglets. Am J Clin Nutr. 2007 February; 85(2):561-9; Wang B., Sialic acid is an essential nutrient for brain development and cognition. Annu Rev Nutr. 2009; 29:177-222; Bode, L., Human milk oligosaccharides: prebiotics and beyond. Nutr Rev. 2009 November; 67 Suppl 2:S183-91). Breast milk, in fact, has been shown to comprise much higher levels of sialic acid content than that of formula and evidence suggests that children fed breast milk develop higher IQ levels that children fed formula (Wang, B. et al., Concentration and distribution of sialic acid in human milk and infant formulas. Am J Clin Nutr. 2001 October; 74(4):510-5; Morgan, B. L. et al., A possible relationship between brain N-acetylneuraminic acid content and behavior. Proc Soc Exp Biol Med. 1979 September; 161(4):534-7; Anderson, J. W. et al., Breast-feeding and cognitive development: a meta-analysis. Am J Clin Nutr. 1999 October; 70(4):525-35).

In view of the importance of sialic acids for nutritional as well as therapeutic applications, some embodiments of the present invention provide methods, compounds and/or compositions that may modulate levels of sialic acids in subjects, including, but not limited to human subjects.

### Compositions of the Invention

In some embodiments, the present invention provides nutraceuticals as well as compositions that comprise nutraceuticals. As used herein, the term nutraceutical refers to an agent that may be eaten or otherwise consumed by a subject to provide a beneficial effect to the subject. As used herein, the term “nutraceutical composition” refers to a composition comprising at least one nutraceutical and at least one other component. In some embodiments, nutraceutical compositions of the present invention comprise carbohydrates. In some embodiments, such carbohydrates further comprise nitrogen. In some embodiments, carbohydrate components of nutraceutical compositions may comprise one or more cyclic hemiacetal ring structures. In some embodiments, carbohydrate components of nutraceutical compositions may comprise one or more monosaccharide. In some embodiments, nutraceutical compositions of the present invention may comprise at least one glycan. As used herein, the term “glycan” refers to a polysaccharide comprising a polymeric chain of two or more monosaccharides. Within a glycan, monosaccharide monomers may all be the same or they may differ. Common monomers include, but are not limited to trioses, tetroses, pentoses, glucose, fructose, galactose, xylose, arabinose, lyxose, allose, altrose, mannose, gulose, iodose, ribose, mannoheptulose, sedoheptulose and talose. Amino sugars may also be monomers within a glycan. Glycans comprising such sugars are herein referred to as aminoglycans. Amino sugars, as used herein, are sugar molecules that comprise an amine group in place of a hydroxyl group or a sugar derived from such a sugar. Examples of amino sugars include, but are not limited to glucosamine, galactosamine, N-acetylglucosamine, N-acetylgalactosamine, sialic acids [including, but not limited to, N-acetylneuraminic acid (Neu5Ac) and N-glycolylneuraminic acid (Neu5Gc)] and L-daunosamine.

In some embodiments, nutraceutical compositions of the present invention may comprise sialic acids. N-acetylneuraminic acid (Neu5Ac) and N-glycolylneuraminic acid (Neu5Gc) are the major sialic acids on mammalian cell surfaces. Of these, Neu5Ac is naturally produced in humans. Neu5Gc is naturally produced in most mammals with the exception of humans due to a mutation in the cytidine monophosphate (CMP)-N-acetylneuraminic acid hydroxylase (CMAH) gene responsible for CMP-Neu5Gc production from CMP-Neu5Ac. Neu5Gc in humans is in fact immunogenic with nearly all humans expressing anti-Neu5Gc antibodies. Despite a lack of production, most human systems comprise some level of Neu5Gc due to dietary intake. These foreign products are subsequently incorporated into human glycoproteins.

In some embodiments, nutraceutical compositions of the present invention comprise glycoconjugates. As used herein, the term “glycoconjugate” refers to any entity comprising a glycan moiety. In some embodiments, glycoconjugates are glycolipids. As used herein, the term “glycolipid” refers to a class of lipids wherein a carbohydrate moiety is covalently attached. In some embodiments, carbohydrate moieties present on glycolipids comprise glycans. In some embodiments, lipid components of glycolipids comprise ceramide moieties. Examples of glycolipids contemplated as targets of the present invention include, but are not limited to glyceroglycolipids (including, but not limited to galactolipids and sulfolipids), glycosphingolipids (including, but not limited to cerebrosides (e.g., galactocerebrosides, glucocerebrosides and sulfatides), gangliosides, globosides and glycophosphosphingolipids) and glycosylphosphatidylinositols. When located within cell membranes, glycan moieties of glycolipids are located on the extracellular side of the membrane where they may interact with other cells as well as cell signaling ligands (Maccioni, H. J. et al., Organization of the synthesis of glycolipid oligosaccharides in the Golgi complex. FEBS Lett. 2011 Jun. 6; 585(11):1691-8).

In some embodiments, nutraceutical compositions of the present invention may comprise glycoproteins and/or proteoglycans. Glycoproteins refer to any proteins that are covalently bonded with glycans. Proteoglycans are a class of proteins that are heavily glycosylated with glycans that often carry a negative charge. This property makes them very hydrophilic and important components of connective tissue.

### Functional Foods

In some embodiments, nutraceutical compositions of the present invention may comprise one or more functional agents. As used herein, a “functional agent” is an entity which exhibits or promotes a property and/or activity by which it is characterized. In some embodiments, a functional agent may exhibit or actively promote a specific function in a subject administered with such an agent. In some embodiments, functional agents are components of functional foods. As used herein, the term “functional food” refers to a food or beverage comprising one or more functional agents. In some embodiments, functional foods naturally comprise one or more functional agents and in other embodiments, functional foods are supplemented, fortified or otherwise modified to possess one or more functional agents or a higher level of one or more functional agents. In some embodiments, functional agents of the present invention are amino sugars, including, but not limited to sialic acids (e.g. Neu5Ac and/or Neu5Gc). In some embodiments, functional agents of the present invention may be glycans comprising sialic acids. In some embodiments, functional agents of the present invention may be glycoproteins comprising sialic acids. Functional foods comprising high levels of sialic acids may be used to provide a health benefit to a subject directly or may provide an indirect health benefit. In some embodiments, functional foods comprising Neu5Ac are used to provide a subject with Neu5Ac for health improvement. In some embodiments, functional foods comprising Neu5Ac are provided to a subject to reduce or eliminate the presence of an alternative form of sialic acid (e.g. Neu5Gc). In some embodiments, components of a functional food are modified to comprise sialic acid (e.g. Neu5Ac and/or Neu5Gc), glycans comprising sialic acid and or glycoproteins comprising sialic acids.

In some embodiments, functional foods may comprise combinations of functional agents. Such functional agents may provide a similar function, provide independent functions or may provide synergistic functions. In some embodiments, functional foods may comprise a combination comprising high levels of Neu5Ac as well as one or more marketed or generic functional agents (e.g. anti-inflammatory agents). In some embodiments, foods are converted to functional foods through the addition of one or more functional agents (such as sialic acid) through sprinkling, mixing and/or coating the food with such functional agents. In some embodiments, foods are converted to functional foods through the addition of one or more functional agents by chemical reaction.

Functional agents and/or functional foods may come in various formats, including, but not limited to liquid, dry, solid, powder, granules, flakes, paste, gel, etc. Functional foods may comprise or be prepared in any number of familiar food or beverage formats including, but not limited to fruits, vegetables, breads, pastas, cereals, crackers, meats, meat substitutes, eggs, egg products, oils, lard, dairy products (e.g. milk, cheese, yogurt, cream, etc,) deserts (e.g. cookies, cakes, cup cakes, pies, puddings, ice cream, candies, chocolates, etc.) smoothies, dips, sauces and condiments.

In some embodiments, functional foods comprise medical foods. As used herein, the term “medical food” refers to a food item specially formulated for the dietary management of a disease or disorder in a subject. Such diseases or disorders may include, but are not limited to cardiovascular disease, cancer, metabolic disorders, malnutrition, dehydration and inflammation. Medical foods may comprise functional agents that function to reduce, ameliorate, eliminate and/or reverse a disease or disorder in a subject. Typically, such functional agents cannot be obtained through a normal diet (e.g. a diet that does not include one or more medical food). In some embodiments, human subjects are administered medical foods under medical supervision. In some embodiments, medical foods may comprise one or more anti-inflammatory agents. Anti-inflammatory agents may include, but are not limited to steroidal anti-inflammatory agents and non-steroidal anti-inflammatory agents. Examples of steroidal anti-inflammatory agents include glucocorticoids. Examples of non-steroidal anti-inflammatory agents include, but are not limited to sialic acid (e.g. Neu5Ac), cyclooxygenase inhibitors (e.g. aspirin, ibuprofen, naproxen) and anti-inflammatory peptides.

### Edible Bird's Nest

As used herein, the term “edible bird's nest” or “EBN” refers to materials from nests of the Swiftlet, a cliff nesting bird with habitats in the southeast regions of Asia as well as the Pacific Islands. EBN is a functional food that has been a component of traditional medicines in Asia for hundreds of years where it is used to treat a variety of illnesses as well as to provide general nourishment and slow the aging process. The component of EBN believed to be responsible for its health and wellness benefits is the dried saliva of the male Swiftlet, deposited during nest construction (Yu-Qin, Y. et al., Determination of edible bird's nest and its products by gas chromatography. J Chromatogr Sci. 2000 January; 38(1):27-32). EBN has been shown to comprise from about 0.1% to about 1.5% lipid, from about 60% to about 65% protein and from about 25% to about 30% carbohydrate (Marcone, M. F., Characterization of the edible bird's nest the “Caviar of the East”. Food Research Intl. 2005 December; 38(10):1125-34). Among carbohydrate components, EBN comprises a high level of sialic acid (typically from about 5% to about 15%). In some embodiments, EBN comprises at least 5%, at least 6%, at least 7%, at least 8%, at least 9%, at least 10%, at least 11%, at least 12%, at least 13%, at least 14% or at least 15% sialic acid. Since birds are unable to synthesize Neu5Gc, the sialic acid present is predominantly Neu5Ac. Other carbohydrate components may include, but are not limited to galactosamine (from about 2% to about 15%), glucosamine (from about 2% to about 15%), galactose (from about 5% to about 30%) and fucose (from about 0.1% to about 5%). At the elemental level, EBN may comprise from about 500 to about 1000 parts per million (ppm) sodium, from about 50 to about 250 ppm potassium, from about 500 to about 1500 ppm calcium, from about 250 ppm to about 750 ppm magnesium, from about 25 to about 75 ppm phosphorus and from about 20 to about 80 ppm iron. Fatty acids present in EBN may include palmitic acid (from about 10% to about 40%), steric acid (from about 10% to about 40%), linoleic acid (from about 10% to about 40%) and linolenic acid (from about 10% to about 40%). In some embodiments of the present invention, nutraceutical compositions comprise one or more components of EBN. In some embodiments, nutraceutical compositions of the present invention comprise synthetic compositions engineered to comprise one or more components of EBN. Such nutraceutical compositions may comprise varying percentages of EBN components.

### Glycomacropeptide

As used herein, the term “glycomacropeptide” or “GMP” refers to a 64 amino acid protein (as well as variants,) derived from the protein k-casein, found in milk, which is hydrolyzed by chymosin and remains in the liquid whey formed during the cheese-making process. Chymosin digestion divides k-casein protein into a larger, para-k-casein, and a small GMP protein (Keogh, J. B. et al., The effect of meal replacements high in glycomacropeptide on weight loss and markers of cardiovascular disease risk. Am J Clin Nutr. 2008 June; 87(6):1602-5). Amino acids 106 to 169 of k-casein make up GMP (LaClair, C. E. et al., 2009. J Food Sci. 74(4): E199-E206.) Exact sequences and phosphorous content may differ depending on the k-casein variant being cleaved. While only 8 kilo Daltons (kDa) in size, the GMP core protein is highly glycosylated giving it an actual molecular weight between 25 and 30 kDa. GMP comprises up to four sialic acid-galactose-N-acetyl galactosamine carbohydrate chains that can be bonded to one or more threonine residues (Rojas, E. et al., 2013. Food Sci. Technol. 33(1): 14-20.) Such residues, may include residues 121, 131, 133, 136, 142 and 165 (corresponding to positions on the un-cleaved k-casein precursor.) Bovine-derived cheese whey comprises from about 10% to about 30% GMP. GMP may be referred to in the art by a number of synonymous terms and abbreviations, including, but not limited to caseinmacropeptide, caseinglycomacropeptide (cGMP), casein-derived peptide (CDP) and caseinglycopeptide (CGP.)

GMP is a functional agent in medical food used to treat subjects with Phenylketonuria (PKU). Individuals with PKU have a defect in metabolism of the amino acid phenylalanine. These individuals require a special diet that is low in phenylalanine Pure GMP is phenylalanine-free making it an ideal source of amino acids for such individuals. Nutraceutical compositions comprising GMP may also be useful in controlling intestinal health, dental health as well as other diseases, disorders and/or conditions (Kawasaki, et al., 1993; Beucher, et al.; Yvon et al., 1994; El Salam, et al., 1996; Dziuba et al., 1996.)

Commercial sources of GMP are available and vary slightly in sialic acid content and purity. Purity of commercially available GMP ranges from about 70% to about 100%. The sialic acid content of commercially available GMP ranges from about 3% to about 10% with from about 98% to about 99.5% of the sialic acid comprising Neu5Ac. The cost of commercially available GMP ranges from about $20/pound to at least $50/pound.

In some embodiments, GMP may be hypersialylated. As used herein, the term “hypersialylated” refers to an entity comprising an excess of sialic acid in comparison to one or more entities comprising a baseline level of sialic acid. In some embodiments, hypersialylated GMP may be isolated from one or more natural sources. In some embodiments, GMP may be synthetically enriched with sialic acids. In other cases, GMP may be hypersialylated with Neu5Ac and/or depleted of Neu5Gc. In some embodiments, GMP may comprise at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29% or at least 30% sialic acid (Neu5Ac and/or Neu5Gc.)

In some embodiments, GMP from a given source may be characterized as hypersialylated in relation to GMP from a different source. In some embodiments, porcine-derived GMP may be hypersialylated in comparison to GMP derived from other sources (e.g. chickens, cows and/or humans.)

### Avian Eggs

Birds do not produce Neu5Gc and therefore their meat and eggs provide a predominantly Neu5Gc-free source of sialic acids. Egg whites are considered to be nutritious due to the presence of high levels of protein with low levels of lipid. Egg whites also comprise Neu5Ac with the overall percentage varying depending on the species from which the egg was derived. Chicken egg whites comprise from about 0.05% to about 1% Neu5Ac, while egg whites from turkey eggs comprise from about 0.5% to about 2% Neu5Ac and emu egg whites comprise from about 1% to about 5% Neu5Ac.

In some embodiments, nutraceuticals of the present invention may comprise egg-derived sialic acid and/or sialylated glycoproteins. Some such glycoproteins may comprise from about 0.5% to about 10% or more sialic acid. In some embodiments, egg-derived sialylated glycoproteins of the invention may comprise at least 0.01%, at least 0.05%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29% or at least 30% sialic acid (Neu5Ac and/or Neu5Gc.) Eggs from any organisms may be useful for this purpose, including, but not limited to eggs obtained from avian species, reptiles, amphibians and fish. In some isolations, chicken eggs may be used. Preparation of free sialic acid and/or sialylated glycoproteins may be carried out using chalaza, egg yolk membrane and/or egg yolk that has been delipidated (Seko, A. et al., Biochimica et Biophysica Acta. 1997. 1335(1-2):23-32.). Such isolations may be carried out through the use of acid hydrolysis and/or protease digestion as described herein below. Additionally, a number of patents using egg yolk as a source for sialic acid and/or sialylated glycoproteins are available.

U.S. Pat. No. 5,233,033 (the contents of which are incorporated herein by reference in their entirety) is directed towards a method for isolating sialic acid and/or sialylated glycoproteins, comprising hydrolysis of delipidated egg yolks and a method for producing purified sialic acid, comprising desalting a solution comprising sialic acid obtainable by delipidated egg yolk hydrolysis, anion exchange resin adsorption of sialic acid and then eluting said sialic acid.

JP Pat. No 08266255 discloses oligosaccharide derivatives comprising sialic acid that may be obtained from chicken egg yolk upon protease-dependent hydrolysis. Such protease-treatment reportedly frees sialylated oligosaccharides; however, it is unclear whether or not all amino acids are removed from such oligosaccharides, or whether residual amino acids may still be present. Another patent JP Pat. No 06245784 discloses enzymatic preparation of compositions comprising sialic acid and/or derivatives. Such compositions may be manufactured by enzymatic processing of delipidated egg yolk (e.g. with proteases), polymer ingredient removal by ultra-filtration of water-soluble fractions, and compound desalting. According to such procedures, egg yolk powder was combined with EtOH to yield delipidated egg yolk. Treatment of 1 ton of delipidated egg yolk with Protease A was carried out in water at 50° C. for 8 h, followed by ultrafiltration and desalting to yield 300 kg of a composition comprising 7.5% free sialic acid and 75% sialyl glycopeptides.

Methods for comprehensive processing and use of poultry eggs are presented in CN Pat. No 1511465. These methods relate to technologies of producing various products, including sialic acids. According to the invention, poultry eggs are washed, crushed and separated in order to individually obtain egg shells, egg whites and yolks. Yolks are processed into sialic acids and lecithin by combining with water, adjusting the pH, carrying out hydrolysis, conducting ion exchange separation, spray drying, phase separation as well as other steps.

### Other Glycoproteins Comprising Sialic Acid

In some embodiments, nutraceutical compositions of the present invention may comprise one or more glycoproteins comprising sialic acid. Such glycoproteins may include, but are not limited to mucins (e.g. submaxillary mucin, salivary mucin), blood/serum glycoproteins, fibrinogen, alpha-1-antitrypsin, antibodies, components of the major histocompatibility complex (MHC), connective tissue, integrins and/or any other proteins capable of becoming glycosylated.

### Proteins and Variants

In some embodiments, nutraceutical compositions of the present invention may comprise proteins and/or variants thereof. Such proteins may exist as a whole polypeptide, a plurality of polypeptides or fragments of polypeptides, which independently may be encoded by one or more nucleic acids, a plurality of nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the nutraceutical compositions of the invention may comprise naturally occurring amino acids and as such may be considered to be proteins, peptides, polypeptides, or fragments thereof. Alternatively, the nutraceutical compositions may comprise both naturally and non-naturally occurring amino acids.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

In some embodiments, the present invention contemplates nutraceutical compositions comprising amino acid based components including variants and derivatives. These include substitutional, insertional, deletion and covalent variants and derivatives. As such, included within the scope of this invention are components of nutraceutical compositions comprising substitutions, insertions, additions, deletions and/or covalent modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions.

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Isotopic Variations

In some embodiments, nutraceutical compositions of the present invention may comprise one or more atoms that are isotopes. As used herein, the term “isotope” refers to a chemical element that has one or more additional neutron. In one embodiment, compounds of the present invention may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. The nutraceutical compositions may be deuterated in order to change a physical property of the compound, such as stability, or to allow the compounds to be used in diagnostic and experimental applications.

### Conjugates and Combinations

In some embodiments of the present invention, nutraceutical compositions may be complexed, conjugated or combined with one or more homologous or heterologous molecules. As used herein, “homologous molecule” means a molecule which is similar in at least one of structure or function relative to a starting molecule while a “heterologous molecule” is one that differs in at least one of structure or function relative to a starting molecule. Structural homologs are therefore molecules which are substantially structurally similar. They can be identical. Functional homologs are molecules which are substantially functionally similar. They can be identical.

In some embodiments, Nutraceutical compositions of the invention may comprise conjugates. Such conjugates of the invention may comprise a naturally occurring substance or ligand, such as a protein [e.g., human serum albumin (HSA) or globulin], lipoprotein [e.g., low-density lipoprotein (LDL), high-density lipoprotein (HDL), very low density lipoprotein (VLDL) or intermediate density lipoprotein (IDL)], a carbohydrate (e.g., a dextran, pullulan, chitin, chitosan, inulin, cyclodextrin or hyaluronic acid) or a lipid. The ligand may also be a recombinant or synthetic molecule, such as a synthetic polymer, e.g., a synthetic polyamino acid, an oligonucleotide (e.g. an aptamer). Examples of polyamino acids include polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

The conjugates can also include targeting groups, e.g., a cell or tissue targeting agent or group, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers.

The targeting group can be any ligand that is capable of targeting a specific receptor. Examples include, without limitation, folate, N-acetylgalactosamine, galactose, mannose, mannose-6-phosphate, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

In still other embodiments, nutraceutical compositions may comprise one or more cell penetrating polypeptides. In some embodiments, such cell penetrating polypeptides may be covalently conjugated to one or more component of nutraceutical compositions. In some embodiments, cell-penetrating peptides may also include a signal sequence. In some embodiments, conjugates of the invention can be designed to have increased stability; increased cell transfection; and/or altered biodistribution (e.g., targeted to specific tissues or cell types).

Conjugating moieties may be added to nutraceutical compositions to allow for labeling or flagging. Such tagging/flagging molecules include, but are not limited to ubiquitin, fluorescent molecules, human influenza hemaglutinin (HA), c-myc (a 10 amino acid segment of the human protooncogene myc with sequence EQKLISEEDL), histidine (His), flag (a short peptide of sequence DYKDDDDK), glutathione S-transferase (GST), V5 (a paramyxovirus of simian virus 5 epitope), biotin, avidin, streptavidin, horse radish peroxidase (HRP) and digoxigenin.

In some embodiments, nutraceutical compositions may be combined with one another or other molecules in the treatment of diseases and/or conditions.

### Nucleic Acids

The present invention embraces nucleic acid molecules. In some embodiments, nucleic acids encode one or more components of nutraceutical compositions. Such nucleic acid molecules include, without limitation, DNA molecules, RNA molecules, polynucleotides, oligonucleotides, mRNA molecules, vectors, plasmids and the like. The present invention also embraces cells programmed or generated to express nucleic acid molecules encoding nutraceutical composition components.

### Methods and Uses

Nutraceutical compositions of the present invention may be used according to a number of different methods. In some embodiments, nutraceutical compositions may be used as part of a method of reducing or eliminating the level of one or more sialic acids in a subject. Such sialic acids may include, but are not limited to Neu5Ac and/or Neu5Gc. According to this method, nutraceutical compositions may be administered orally. In some cases, sialic acid levels may be reduced or eliminated in one or more specific tissues. Such tissues may include, but are not limited to tissues of the trachea, lungs, skin, intestine, heart and aorta.

Also provided are methods of evaluating the ability of nutraceutical compositions to reduce or eliminate sialic acids (e.g. Neu5Ac and/or Neu5Gc) from a subject. According to such methods, subject samples may be taken periodically from a subject being administered a nutraceutical composition of the invention and sialic acid levels may be determined and compared among samples taken. With such methods, subject samples may be obtained minutes, hours, days or weeks apart. In some cases, samples are obtained from about 1 week to about 52 weeks apart (including from about 1 week to about 12 weeks, from about 2 weeks to about 24 weeks or from about 4 weeks to about 48 weeks apart.) According to further methods of evaluating the ability of nutraceutical compositions to reduce or eliminate sialic acds from a subject, the level of anti-sialic acid antibodies may be obtained (e.g. anti-Neu5Gc antibodies.)

In some embodiments, nutraceutical compositions of the present invention may be administered as part of a method to incorporate sialic acid (e.g. Neu5Ac and/or Neu5Gc) into one or more tissues of a subject. Such tissues may include, but are not limited to tracheal, lung and/or skin tissue. In some cases, nutraceutical compositions may be administered as part of a method of increasing the level of sialic acid (e.g. Neu5Ac and/or Neu5Gc) in one or more tissues of a subject. Such tissues may include, but are not limited to tracheal, lung and/or skin tissue.

### Therapeutics

In some embodiments, nutraceutical compositions of the present invention may be used as therapeutics for the treatment of one or more disease, disorder and/or condition.

### Oncology-Related Therapeutics

Chronic inflammation, such as that induced in humans by dietary consumption of food products comprising Neu5Gc, is linked to the development of cancer. In fact, chronic inflammation may contribute to all major stages involved with tumor progression including, but not limited to cell transformation, primary tumor growth and metastasis.

To date, Neu5Gc has been detected in glycoconjugates from a number of human cancer tissues including, but not limited to colon cancer, retinoblastoma tissue, melanoma, breast cancer and yolk sac tumor tissue. In some embodiments of the present invention, administration of nutraceutical compositions of the present invention may prevent, reduce and/or reverse the effects of these forms of cancer as well as other forms of cancer, not specifically listed here, characterized by the presence of cancer cells comprising Neu5Gc.

### Immune-Related Therapeutics

Many bacterial glycans are known to comprise sialic acid. In some cases, such glycans allow bacteria to evade the innate immune system of hosts, including, but not limited to humans. In one example, bacterial glycans inhibit alternate complement pathway activation through factor H recognition. In another example, bacterial glycans mask underlying residues that may be antigenic. Some bacterial glycans participate in cell signaling events through activation of inhibitory sialic acid binding Ig-like lectins (Siglecs) that dampen the immune response to entities comprising certain sialylated moieties (Chen, X. et al., Advances in the biology and chemistry of sialic acids. ACS Chem Biol. 2010 Feb. 19; 5(2):163-76). In some embodiments, nutraceutical compositions of the present invention may be used to prevent, reduce and/or reverse the effects of immune complications related to bacterial glycans.

Due to the foreign nature of Neu5Gc as described herein, some Neu5Gc glycans are immunogenic resulting in inflammation and immune related destruction of cells and other entities where these glycans may be expressed. Such autoimmune destruction may be pathogenic. In some embodiments, nutraceutical compositions of the present invention may be used to treat patients suffering from autoimmune disorders related to Neu5Gc glycans.

In some embodiments, nutraceutical composition of the present invention may modulate inflammatory biomarker levels in a subject. In some embodiments, inflammatory biomarkers may include, but are not limited to interferon gamma (IFNγ,) interleukin (IL)-1β, IL-2, IL-4, IL-5, IL-6, IL-10, IL-12p70, IL-13, IL-17A, keratinocyte chemoattractant (KC), monocyte chemotactic protein 1 (MCP-1), tumor necrosis factor (TNF) α, serum amyloid A (SAA) and haptoglobin. Of these, IL-10, IL-13 and IL-4 are involved in anti-inflammatory processes, while INFγ, IL-1β, IL-2, IL-5, IL-6, IL-12p70, IL-17A, KC, MCP-1, TNFα, SAA and haptoglobin are involved in elevated inflammation.

In some embodiments, the present invention provides methods of evaluating the ability of nutraceutical compositions to modulate the level of one or more inflammatory biomarkers in a subject. Such methods may comprise one or more of the steps: 1) administering a nutraceutical composition to a subject, 2) obtaining a first sample from the subject, 3) obtaining a second sample from the subject from about 1 week to about 12 weeks after obtaining a first sample, 4) determining the level of one or more inflammatory biomarkers (e.g. IFNγ, IL-1β, IL-2, IL-4, IL-5, IL-6, IL-10, IL-12p70, IL-13, IL-17A, KC, MCP-1, TNFα, SAA and/or haptoglobin) in the first sample and the level of one or more inflammatory biomarkers in the second sample, and 5) comparing the level of the inflammatory biomarkers in the second sample to the level of the inflammatory biomarkers in the first sample.

### Cardiovascular Therapeutics

Chronic inflammation is implicated in cardiovascular disease. In some embodiments, nutraceutical compositions of the present invention may be used to treat cardiovascular disease. In some embodiments, nutraceutical compositions may function to reduce levels of Neu5Gc in cardiovascular tissues including, but not limited to the heart, arteries (including, but not limited to the aorta) and veins.

### Metabolic Therapeutics

In some embodiments, nutraceutical compositions of the present invention may be useful for the treatment of subjects afflicted with one or more metabolic diseases, disorders and/or conditions. In such embodiments, nutraceutical composition may be used to prevent, reduce or reverse the effects of such metabolic diseases, disorders and/or conditions.

### Phenylketonuria

Individuals with PKU have a genetic defect in metabolism of the amino acid phenylalanine [MedlinePlus (Internet). Bethesda (Md.): National Library of Medicine (US); (updated 2013 May 24). Phenylketonuria; (updated 2013 Mar. 22; reviewed 2013 May 27; cited 2013 May 27); (about 2 p.). Available from: http://www.nlm.nih.gov/medlineplus/ency/article/001166.htm.] These individuals lack the enzyme phenylalanine hydroxylase (used to degrade phenylalanine) and require a special diet that is low in phenylalanine. In some embodiments, nutraceutical compositions of the present invention may be useful for the treatment of subjects afflicted with PKU. In such embodiments, nutraceutical compositions disclosed herein may prevent, reduce and/or reverse one or more symptoms related to PKU including, but not limited to cognitive delays, small head size, hyperactivity, uncontrollable arm and/or leg movements, seizures, skin rashes, tremors and/or unusual hand positioning.

### Nutritional Applications

### Dietary Supplements

In some embodiments, nutraceuticals of the present invention may be dietary supplements. As used herein the term “dietary supplement” refers to an ingested substance comprising one or more ingredients intended to supplement the diet of a subject by increasing the total dietary intake of such ingredients. Dietary supplements are typically distinct from conventional food or as sole items of a meal or diet. In some embodiments, dietary supplements may be ingested in the form of tablets, capsules, powders, softgels, gelcaps, and/or in liquid form (typically comprising about 3 ounces or less of such liquid.) Dietary supplements may effect bodily structure and/or function.

### Pre- and Post-Natal Applications

Sialic acids are an essential component of brain gangliosides and sialylated glycoproteins, which play critical roles in mediating cell-to-cell interactions important for neuronal outgrowth, synaptic connectivity and memory formation. A diet rich in sialic acids has been shown to increase the levels of learning-related genes and enhance learning and memory. Human breast milk contains an exceptionally high level of glycoconjugates comprising sialic acid (predominantly Neu5Ac). Conventional infant formula; however, contains less than 25% of the sialic acid found in human breast milk. Therefore, the addition of sialic acid to infant formula may ultimately benefit brain development in utero (Wang, B., Molecular mechanism underlying sialic acid as an essential nutrient for brain development and cognition. Adv Nutr. 2012 May 1; 3(3):465S-72S). In some embodiments, nutraceutical compositions may be added to or combined with infant formula to provide one or more of the benefits associated with sialic acid consumption in infants.

### Fitness Applications

In some embodiments, nutraceutical compositions of the present invention may be administered to promote and/or enhance health and/or fitness in one or more subjects.

### Processed Meat Products

In some embodiments, functional agents, as described herein, may be combined with one or more meats in the production of functional foods that are processed meat products. As used herein, the term “processed meat product” refers to meat-based food that undergoes one or more preparative steps. Such preparative steps may include, but are not limited to chopping, grinding, mincing, salting, curing, tenderizing, heating, cooking, drying, dehydrating, fermenting, liquefying, extruding, freezing and compressing. Processed meat products can be derived from any edible animal (e.g. cows, pigs, goats, sheep, chickens, fish, etc.) and can be prepared in a variety of formats including, but not limited to patties, nuggets, sausages and loaves. Exemplary processed meat products may include, but are not limited to hamburgers, hotdogs, sausages, salamis, meatballs, cold cuts, bologna, chicken nuggets, chicken fingers, kebabs, hams and dry sausages.

In some cases, functional agents may be combined with finely textured meats. As used herein, the term “finely textured meat” refers to a processed meat product made up of tiny bits of meat obtained from meat trimmings. Finely textured meat is typically produced through heating (to liquefy fats in the meat,) followed by centrifugation to separate the meat from the fat. Finely textured meat is often further treated to reduce the presence of microbes. Such treatments may include exposing the meat with ammonia gas or citric acid. Examples of finely textured meat include lean finely textured beef (LFTB) and boneless lean beef trimmings (BLBT.)

The amount of one or more functional agents combined with one or more meats to generate a processed meat product may be varied to achieve a desired concentration of the one or more functional agents or a component of such functional agents (e.g. sialic acid, including, but not limited to Neu5Ac and/or Neu5Gc.) In some embodiments, processed meat products may be formulated to comprise from about 0.0001 to about 99.999 weight % of a functional agent. In some cases, processed meat products may comprise from about 0.0001 to about 0.01, from about 0.001 to about 0.1, from about 0.005 to about 0.2, from about 0.02 to about 0.5, from about 0.5 to about 2.0, from about 1.0 to about 10, from about 2 to about 20, from about 5 to about 25, from about 10 to about 30, from about 15 to about 45, from about 20 to about 50, from about 40 to about 60, from about 50 to about 75, from about 60 to about 80, from about 75 to about 99 and from about 90 to about 99.9 weight percent of a functional agent. In some cases, functional agents may be combined with one or more meats on a functional agent weight/meat weight basis (e.g. g/g, mg/g, mg/kg, g/kg, ounces per ounces, ounces per pound, pound per pound, pounds per ton, etc.) In some cases, functional agents may be combined with one or more meats on a functional agent weight/meat volume (e.g. μg/ml, mg/ml, g/ml, mg/L, g/L, kg/L, etc,) functional agent volume/meat weight (e.g. μl/mg, μl/g, ml/mg, ml/g, ml/kg, L/kg, fluid ounces/pound, gallons/ton, etc.) or functional agent volume per meat volume (e.g. μl/μl, μl/ml, ml/ml, ml/L, L/L, fluid ounces/fluid ounces, fluid ounces/gallon, gallons/gallon, etc.) basis. In some cases, functional agents may be combined with one or more meats by ratio. Such functional agent:meat ratios may include from about 100:1 to about 1:1 (e.g. about 90:1, about 80:1, about 70:1, about 60:1, about 50:1, about 40:1, about 30:1, about 20:1, about 10:1, about 5:1, about 4:1, about 3:1, about 2:1, about 1.5:1, about 1.2:1, about 1.1:1, etc.) or from about 1:1 to about 1:10⁶ (e.g. about 1:1.1, about 1:1.2, about 1:1.5, about 1:2, about 1:5, about 1:10, about 1:20, about 1:30, about 1:40, about 1:50, about 1:60, about 1:70, about 1:80, about 1:90, about 1:100, about 1:500, about 1:1000, about 1:2000, about 1:3000, about 1:5000, about 1:10000, about 1:20000, about 1:30000, about 1:50000, about 1:100000, about 1:200000, about 1:500000, etc.)

Processed meat products of the present invention are produced by combining one or more meats with one or more functional agents comprising sialic acid. Such functional agents may comprise Neu5Ac, Neu5Gc, or both Neu5Ac and Neu5Gc. In some instances, functional agents comprising sialic acid may comprise a total sialic acid content in which more than 50% of the total sialic acid content is made up of Neu5Ac. Functional agents comprising sialic acid may include sialic acid-enriched functional agents, in which the functional agents have been modified to comprise sialic acid and/or increased levels of sialic acid

Processed meat products of the invention may be prepared by combining one or more meats with GMP. In some cases, the GMP combined is enriched with Neu5Ac and/or depleted of Neu5Gc to provide a favorable sialic acid profile for human health. Processed meat products that have been combined with GMP may be used to, in some cases, to modulate inflammatory biomarker levels in a subject. In some embodiments, such inflammatory biomarkers may include, but are not limited to interferon gamma (IFNγ,) interleukin (IL)-1β, IL-2, IL-4, IL-5, IL-6, IL-10, IL-12p70, IL-13, IL-17A, keratinocyte chemoattractant (KC), monocyte chemotactic protein 1 (MCP-1), tumor necrosis factor (TNF) α, serum amyloid A (SAA) and haptoglobin.

### Veterinary Applications

It is contemplated that nutraceutical compositions of the invention may find utility in the area of veterinary care including the care and treatment of non-human vertebrates. As described herein, the term “non-human vertebrate” includes all vertebrates with the exception of Homo sapiens, including wild and domesticated species such as companion animals and livestock. Non-human vertebrates include mammals, such as alpaca, banteng, bison, camel, cat, cattle, deer, dog, donkey, gayal, goat, guinea pig, horse, llama, mule, pig, rabbit, reindeer, sheep water buffalo, and yak. Livestock includes domesticated animals raised in an agricultural setting to produce materials such as food, labor, and derived products such as fiber and chemicals. Generally, livestock includes all mammals, avians and fish having potential agricultural significance. In particular, four-legged slaughter animals include steers, heifers, cows, calves, bulls, cattle, swine and sheep.

### Characterization of Nutraceutical Compositions

In some embodiments, nutraceutical compositions may be characterized by one or more of bioavailability, therapeutic window and/or volume of distribution.

### Bioavailability

Nutraceuticals, when formulated into a composition with a delivery/formulation agent or vehicle as described herein, may exhibit an increase in bioavailability as compared to a composition lacking a delivery agent as described herein. As used herein, the term “bioavailability” refers to the systemic availability of a given amount of nutraceuticals administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference.

The Cₘₐₓ value is the maximum concentration of the compound achieved in the serum or plasma of a mammal following administration of the compound to the mammal. The C. value of a particular compound can be measured using methods known to those of ordinary skill in the art. The phrases “increasing bioavailability” or “improving the pharmacokinetics,” as used herein mean that the systemic availability of nutraceuticals, measured as AUC, Cₘₐₓ, or Cₘᵢₙ in a mammal is greater, when co-administered with a delivery agent as described herein, than when such co-administration does not take place. In some embodiments, the bioavailability of nutraceuticals can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

Nutraceuticals, when formulated into a composition with a delivery agent as described herein, can exhibit an increase in the therapeutic window of administered nutraceutical compositions as compared to the therapeutic window of administered nutraceutical compositions lacking a delivery agent as described herein. As used herein “therapeutic window” refers to the range of plasma concentrations, or the range of levels of therapeutically active substance at the site of action, with a high probability of eliciting a therapeutic effect. In some embodiments, the therapeutic window of nutraceuticals when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

Nutraceuticals, when formulated into a composition with a delivery agent as described herein, can exhibit an improved volume of distribution (V_{dist}), e.g., reduced or targeted, relative to a composition lacking a delivery agent as described herein. The volume of distribution (V_{dist}) relates the amount of a compound in the body to the concentration of such compounds in the blood or plasma. As used herein, the term “volume of distribution” refers to the fluid volume that would be required to contain the total amount of a compound in the body at the same concentration as in the blood or plasma: V_{dist} equals the amount of a compound in the body/concentration of a compound in blood or plasma. For example, for a 10 mg dose and a plasma concentration of 10 mg/L, the volume of distribution would be 1 liter. The volume of distribution reflects the extent to which a compound is present in the extravascular tissue. A large volume of distribution reflects the tendency of a compound to bind to the tissue components compared with plasma protein binding. In a clinical setting, V_{dist} can be used to determine a loading dose to achieve a steady state concentration. In some embodiments, the volume of distribution of nutraceuticals when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

In some embodiments, nutraceuticals may be combined with one or more pharmaceutically acceptable excipients. Nutraceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, compositions are administered to humans, human patients or subjects. For the purposes of the present disclosure, the phrase “active ingredient” generally refers to nutraceuticals to be delivered as described herein.

Although the descriptions of nutraceutical compositions provided herein are principally directed to nutraceutical compositions which are suitable for administration to humans, it will be understood by the skilled artisan that such compositions are generally suitable for administration to any other animal, e.g., to non-human animals, e.g. non-human mammals. Modification of nutraceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of nutraceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the nutraceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A nutraceutical composition in accordance with the invention may be prepared, packaged, and/or sold in bulk, as a single unit dose, and/or as a plurality of single unit doses. As used herein, a “unit dose” is discrete amount of the nutraceutical composition comprising a predetermined amount of active ingredient. The amount of active ingredient is generally equal to the dosage of active ingredient which would be administered to a subject and/or a convenient fraction of such a dosage such as, for example, one-half or one-third of such a dosage.

Relative amounts of active ingredient, the pharmaceutically acceptable excipient, and/or any additional ingredients in a nutraceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient. In some embodiments, active ingredients are nutraceuticals used to flush Neu5Gc.

### Formulations

Nutraceuticals of the invention can be formulated using one or more excipients to: (1) increase stability; (2) increase cell permeability; (3) permit the sustained or delayed release (e.g., from a formulation of the nutraceutical); and/or (4) alter the biodistribution (e.g., target nutraceuticals to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, formulations of the present invention can include, without limitation, liposomes, lipid nanoparticles, polymers, lipoplexes, core-shell nanoparticles, peptides, proteins, cells transfected with one or more nutraceutical composition components (e.g., for transplantation into a subject) and combinations thereof.

### Excipients

As used herein, the term “excipient” refers to any substance combined with an active ingredient (e.g. sialic acids, functional agents and/or nutraceuticals) before use. In some embodiments, excipients are inactive and used primarily as a carrier, diluent or vehicle for an active ingredient. Various excipients for formulating nutraceutical compositions and techniques for preparing such compositions are known in the art (see Remington: The Science and Practice of Pharmacy, 21^{st} Edition, A. R. Gennaro, Lippincott, Williams & Wilkins, Baltimore, Md., 2006; incorporated herein by reference).

The use of a conventional excipient medium is contemplated within the scope of the present disclosure, except insofar as any conventional excipient medium may be incompatible with a substance or its derivatives, such as by producing any undesirable biological effect or otherwise interacting in a deleterious manner with any other component(s) of nutraceutical compositions.

Formulations of nutraceutical compositions described herein may be prepared by any method known or hereafter developed in the art of pharmacology. In general, such preparatory methods include the step of associating the active ingredient with an excipient and/or one or more other accessory ingredients.

A nutraceutical composition in accordance with the present disclosure may be prepared, packaged, and/or sold in bulk, as a single unit dose, and/or as a plurality of single unit doses.

Relative amounts of active ingredients, excipients, and/or any additional ingredients in a nutraceutical compositions in accordance with the present disclosure may vary, depending upon the identity, size, and/or condition of the subject being treated and further depending upon the route by which nutraceutical compositions may be administered.

In some embodiments, acceptable excipients are at least 95%, at least 96%, at least 97%, at least 98%, at least 99%, or 100% pure. In some embodiments, excipients are approved for use in humans and/or for veterinary use. In some embodiments, excipients are approved by the United States Food and Drug Administration. In some embodiments, excipients are pharmaceutical grade. In some embodiments, excipients meet the standards of the United States Pharmacopoeia (USP), the European Pharmacopoeia (EP), the British Pharmacopoeia, and/or the International Pharmacopoeia.

Acceptable excipients used in the manufacture of nutraceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in nutraceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [Span®60], sorbitan tristearate [Span 65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Food Additives

In some cases, excipients may include one or more food additives. Food additives may include, but are not limited to, acids and acidity regulators, anticaking additives, antifoaming additives, bulking additives, coloring additives (including, but not limited to additives to enhance, replace or preserve color,) antioxidants, emulsifiers, flavor additives (including, but not limited to specific flavors and flavor enhancers,) humectants, preservatives, stabilizers, sweeteners and thickeners. Exemplary acids and acidity regulators may include, but are not limited to Acetic acid, Ammonium adipates, Calcium gluconate, Fumaric acid, Glucono delta-lactone, Hydrochloric acid, Magnesium citrate, Malic acid, Sulfuric acid, Sodium aluminium phosphate, Sodium succinates, Lactic acid, Carbon dioxide, Adipic acid, Ammonium ferric citrate, Ammonium fumarate, Ammonium lactate, Ammonium malate, Calcium fumarate, Calcium lactate, Calcium malates, Citric acid, Ferric ammonium citrate, L(+)-Tartaric acid, Magnesium lactate, Phosphoric acid, Potassium adipate, Potassium citrates, Potassium fumarate, Potassium lactate, Potassium malate, Potassium sodium tartrate, Potassium tartrates, Sodium adipate, Sodium citrates, Sodium fumarate, Sodium lactate, Sodium malates, Sodium tartrates, Succinic acid, Triammonium citrate, 1,4-heptonolactone, Calcium tartrate, Metatartaric acid and Calcium citrates. Exemplary anticaking additives may include, but are not limited to Aluminium silicate, Ammonium polyphosphates, Bentonite, Bone phosphate, Calcium aluminosilicate (calcium aluminium silicate), Calcium ferrocyanide, Calcium polyphosphates, Calcium silicate, Dicalcium diphosphate, Kaolin, Magnesium oxide, Magnesium silicate, Microcrystalline cellulose, Potassium aluminium silicate, Potassium ferrocyanide, Powdered Cellulose, Silicon dioxide, Sodium aluminosilicate (sodium aluminium silicate), Sodium ferrocyanide, Stearic acid, Talc and Magnesium carbonate. Antifoaming additives may include, but are not limited to polyethylene glycol 8000 and polymethylsiloxane. Exemplary antioxidants may include, but are not limited to Delta-tocopherol, Dilauryl thiodipropionate, Distearyl thiodipropionate, Dl-alpha-tocopherol, Dodecyl gallate, Erythorbin acid, Gamma-tocopherol, Glucose oxidase, Octyl gallate, Propyl gallate, Sodium erythorbate, Sodium erythorbin, Tert-butylhydroquinone, Thiodipropionic acid, Tocopherol concentrate (natural), Ascorbyl palmitate, Ascorbyl stearate, Butylated hydroxyanisole (BHA), Butylated hydroxytoluene (BHT), Ascorbic acid (Vitamin C), Calcium ascorbate, Potassium ascorbate, Sodium ascorbate, EDTA, Lecithins, Disodium ethylenediaminetetraacetate, Isopropyl citrates and Oxystearin. Exemplary coloring additives may include, but are not limited to Annatto, Anthocyanins, Astaxanthin, Beta-apo-8′-carotenal (C 30), Beta-apo-8′-carotenic acid ethyl ester, Bixin, Canthaxanthin, Capsanthin, Capsorubin, Carotenes, Alpha-carotene, Beta-carotene, Gamma-carotene, Chocolate Brown HT, Citranaxanthin, Crocetin, Crocin, Cryptoxanthin, Flavoxanthin, Iron oxides and hydroxides, Latolrubine, Lithol Rubine BK, Litholrubine, Lutein, Lycopene, Norbixin, Pigment Rubine, Ponceau 6R, Ponceau SX, Red 2G, Rhodoxanthin, Rubixanthin, Saffron, Sandalwood, Scarlet GN, Violaxanthin, Zeaxanthin, Indanthrene blue RS, Patent blue V, Indigo carmine, Indigotine, Black 7984, Black PN, Brown FK, Caramel I, Caramel II (Caustic Sulfite process), Caramel III (Ammonia process), Caramel IV (Ammonia sulfite process), Carbon black, Vegetable carbon, Brilliant Scarlet 4R, Ponceau 4R, Brilliant blue FCF, Fast green FCF, Allura red AC, Gold, Chlorophylls and Chlorophyllins, Copper complexes of chlorophylls, Green S, Orange GGN, Alkanet, Beet red, Beetroot red, Betanin, carmines, Chrysoine resorcinol, Citrus red 2, Cochineal, Orcein, Orchil, Amaranth, Azorubine, Carmoisine, Erythrosine, Aluminium, Silver, Titanium dioxide, Calcium carbonates, Chalk, Curcumin, Riboflavin (vitamin B2), Turmeric, Yellow 2G, Quinoline Yellow WS, Tartrazine, Sunset Yellow FCF, Ferrous gluconate, Niacin (vitamin B3), nicotinic acid (vitamin B3), Nicotinamide (vitamin B3), stannous chloride, Polyvinylpolypyrrolidone and Tannins Sweeterners may include artificial sweeteners including, but not limited to Acesulfame potassium, Alitame, Aspartame, Cyclamates, Cyclamic acid, Erythritol, Neohesperidin dihydrochalcone, Saccharin and Sucralose. Exemplary emulsifiers may include, but are not limited to Acetic acid esters of mono- and diglycerides of fatty acids, Ammonium phosphatides, Calcium stearoyl lactylate, Choline salts and esters, Citric acid esters of mono- and diglycerides of fatty acids, Crosslinked Sodium carboxymethylcellulose, beta-cyclodextrin, Diacetyltartaric acid esters of mono- and diglycerides of fatty acids, Dioctyl sodium sulfosuccinate, Enzymatically hydrolyzed Carboxymethyl cellulose, Glycerol ester of wood rosin, Glyceryl distearate, Glyceryl monostearate, Lactic acid esters of mono- and diglycerides of fatty acids, Lactylated fatty acid esters of glycerol and propylene glycol, Mixed acetic and tartaric acid esters of mono- and diglycerides of fatty acids, Mono- and diglycerides of Fatty acids, Polyglycerol esters of fatty acids, Polyglycerol polyricinoleate, Polyoxyethylene (40) stearate, Polysorbate 20, Polysorbate 40, Polysorbate 60, Polysorbate 65, Polysorbate 80, Propylene glycol esters of fatty acids, Sodium carboxymethylcellulose, Sodium stearoyl lactylate, Sorbitan monolaurate, Sorbitan monooleate, Sorbitan monopalmitate, Sorbitan monostearate, Sorbitan tristearate, Stearyl tartarate, Sucroglycerides, Sucrose esters of fatty acids, Tartaric acid esters of mono- and diglycerides of fatty acids, Thermally oxidised soya bean oil, Dimethylpolysiloxane, Brominated vegetable oil, Magnesium stearate, Sucrose acetate isobutyrate, Calcium salts of fatty acids, Magnesium salts of fatty acids, Potassium salts of fatty acids, Sodium salts of fatty acids and Polyoxyethylene (8) stearate. Exemplary flavor additives may include, but are not limited to Malt extract, Calcium 5′-ribonucleotides, Calcium diglutamate, Calcium guanylate, Calcium inosinate, Dipotassium guanylate, Dipotassium inosinate, Disodium 5′-ribonucleotides, Disodium guanylate, Disodium inosinate, Ethyl maltol, Glutamic acid, Glycine, Guanylic acid, Inosinic acid, Leucine, Lipases, Magnesium diglutamate, Maltol, Monoammonium glutamate, Monopotassium glutamate, Monosodium glutamate (MSG), Zinc acetate, Thaumatin, Cumin oil/Black seed oil, Juniper berry oil, Cinnamon oil, Walnut oil, Hazelnut oil and Tetrahydrocannabinol. In some cases, one or more agents for flour treatment and/or bleaching may be used. Such agents may include, but are not limited to Azodicarbonamide, Amylases, Benzoyl peroxide, Carbamide, Chlorine dioxide, Chlorine, L-cysteine, Potassium bromate and Calcium sulfate. In some cases, one or more glazing agents may be used. Examples of glazing agents may include, but are not limited to Beeswax, Candelilla wax, Carnauba wax, Paraffins, Refined microcrystalline wax and Shellac. Exemplary humectants may include, but are not limited to Isomalt, Lactitol, Montanic acid esters, Oxidised polyethylene wax, Polydextrose, Propylene glycol, Quillaia extract, Triacetin, Mannitol, Sorbitol, Maltitol, Xylitol and Glycerin. In some cases, one or more mineral salts may be included as a food additive. Examples of mineral salts include, but are not limited to Aluminium ammonium sulfate, Aluminium potassium sulfate, Aluminium sodium sulfate, Aluminium sulfate, Ammonium bicarbonate, Ammonium carbonate, Ammonium chloride, Ammonium hydroxide, Ammonium phosphates, Calcium chloride, Calcium hydroxide, Calcium oxide, Cupric sulfate, Magnesium chloride, Magnesium hydroxide, Potassium bicarbonate, Potassium carbonate, Potassium chloride, Potassium hydroxide, Potassium phosphates, Sodium bicarbonate, Sodium carbonate, Sodium hydroxide, sodium phosphates, Epsom salts, Magnesium sulfate, Magnesium phosphates, Calcium phosphates, Diphosphates, Polyphosphates, Triphosphates, Ammonium sulfate, Sodium sulfite and Potassium sulfate. Exemplary preservatives may include, but are not limited to 2-hydroxybiphenyl, Benzoic acid, Biphenyl, Borax, Boric acid, Calcium benzoate, Calcium disodium EDTA, Calcium formate, Calcium propionate, Calcium sorbate, Dehydroacetic acid, Dimethyl dicarbonate, Diphenyl, Ethylparaben (ethyl para-hydroxybenzoate), Formaldehyde, Formic acid, Gum guaicum, Heptyl p-hydroxybenzoate, Hexamine (hexamethylene tetramine), Lecithin citrate, Lysozyme, Methylparaben (methyl para-hydroxybenzoate), Natamycin, Nisin, Orthophenyl phenol, Phytic acid, Pimaricin, Potassium benzoate, Potassium propionate, Potassium sorbate, Propionic acid, Propylparaben (propyl para-hydroxybenzoate), Sodium benzoate, Sodium dehydroacetate, Sodium ethyl para-hydroxybenzoate, Sodium formate, Sodium methyl para-hydroxybenzoate, Sodium orthophenyl phenol, Sodium propionate, Sodium propyl para-hydroxybenzoate, Sodium sorbate, Sodium tetraborate, Sorbic acid, Thiabendazole, Ammonium acetate, Calcium acetate, Glacial Acetic acid, Potassium acetates, Sodium acetate, Sodium hydrogen acetate, Calcium bisulfite, Calcium hydrogen sulfite, Calcium sulfite, Potassium bisulfite, Potassium hydrogen sulfite, Potassium metabisulfite, Potassium sulfite, Sodium bisulfite (sodium hydrogen sulfite), Sodium sulfite, Sulfur dioxide, Sodium metabisulfite, Potassium nitrate, Potassium nitrite, Sodium nitrate and Sodium nitrite. In some cases, one or more propellant may be used as a food additive. These may include, but are not limited to Argon, Butane, Helium, Isobutane, Nitrogen and Nitrous oxide. Exemplary thickeners may include, but are not limited to Methylcellulose, Acetylated distarch adipate, Acetylated distarch phosphate, Acetylated oxidised starch, Acetylated starch, Acid treated starch, Alkaline treated starch, Arabinogalactan, Bleached starch, Dextrin roasted starch, Distarch phosphate, Enzyme treated starch, Hydroxypropyl distarch phosphate, Hydroxypropyl starch, Konjac, Konjac glucomannate, Konjac gum, Monostarch phosphate, Oxidised starch, Phosphated distarch phosphate, Starch sodium octenylsuccinate, Triethyl citrate, Ethyl methyl cellulose, Hydroxypropyl cellulose, Hydroxypropyl methylcellulose, Methyl ethyl cellulose, Guar gum, Tara gum, Xanthan gum, Gellan gum, Gum arabic, Karaya gum, Propane-1,2-diol alginate, Propylene glycol alginate, Tragacanth, Agar, Alginic acid, Ammonium alginate, Calcium alginate, Carrageenan, Locust bean gum, Potassium alginate, Processed Eucheuma seaweed and Sodium alginate.

Additional compounds that may be used as food additives may include, but are not limited to Abietic acid, Acacia, Acacia vera, Acesulfame, Alcohol, Alfalfa, Allspice, Almond oil, Amaranth oil, Amchur (mango powder), Angelica (Angelica archangelica), Anise, Apricot oil, Argan oil, Asafoetida, Avocado oil, Babassu oil, Baking powder, Baking soda, Balm oil, Balm, lemon, Balsam of Peru, Barberry, Barley flour, Basil, Basil extract, Bay leaves, Ben oil, Berebere, Bergamot, Bergamot (Monarda didyma), Bison grass (Hierochloe odorata), Black cardamom, Black cumin, Black limes, Blackcurrant seed oil, Bolivian Coriander (Porophyllum ruderale), Borage (Borago officinalis), Borage seed oil, Brilliant Black BN-color (brown and black), Bush tomato, Cacao shell, Cachou extract, Cactus root extract, Cadinene, Caffeine, Cajeput oil, Calamus, Calcium ascorbate (Vitamin C), Calcium lactobionate, Calcium pantothenate (Vitamin B5), Calcium peroxide, Camellia oil/Tea oil, Camomile, Candle nut, Canola oil/Rapeseed oil, Caper, Caraway, Cardamom, Carob Pod, Carob pod oil/Algaroba oil, Carrageenan, Carrot Oil, Cashew oil, Cassia, Catechu extract, Celery salt, Celery seed, Chervil, Chicory, Chicory Root Extract, Chile pepper, Chili powder, Chives, Cicely, Cilantro, Cinnamon, Cloves, Coconut oil, Coriander, Coriander seed oil, Corn oil, Corn syrup, Cottonseed oil, Cress, Cumin, Curry leaf, Curry powder, Cyanocobalamin (Vitamin B12), Damiana (Turnera aphrodisiaca, T. diffusa), Dandelion (Taraxacum officinale), Dandelion leaf, Dandelion Root, Decanal, Decanal dimethyl acetal, Decanoic acid, Devil's claw (Harpagophytum procumbens), Dill (Anethum graveolens), Dill seed, Echinacea, Egg, Egg white, Egg yolk, Elderberry, Eleutherococcus senticosus, Epazote (Chenopodium ambrosioides), Ethanol (alcohol), Ethylenediamine tetraacetic acid, Evening primrose, Evening primrose oil, False flax oil made of the seeds of Camelina sativa, Fantesk, Farnesol, Fat, Fennel (Foeniculum vulgare), Fenugreek, Ferrous lactate, File powder, Five-spice powder, Fo-ti-tieng, Fructose, Galangal, Galangal root, Galbanum oil, Gallic acid, Garam masala, Garlic, Garlic extract, Garlic oil, Gelatin/gelatine, Ginger, Ginger oil, Ginger root, Ginseng, Glucitol, Gluconate, Glucose syrup, Glutamate, Gluten, Glycerol, Glyoxylic acid, Grains of paradise, Grape color extract, Grape seed oil, Green tea, Guaranine, Haw bark, Heliotropin, Hemlock oil, Hemp oil, Hesperidin, Hexyl acetate, High fructose corn syrup, Horseradish, Hyssop (Hyssopus officinalis), Indole, Inosinate, Inositol, Insoluble fiber, Intense sweeteners, Inulin, Invert sugar, Invertase, Iron, Iron ammonium citrate, Jamaican jerk spice, Jasmine, Jasmine absolute, Jiaogulan (Gynostemma pentaphyllum), Juniper, Juniper berry, Juniper extract, Kaffir Lime Leaves, Kapok seed oil, Kelp, Kokam, Kola nut extract, Lactose, Larch gum, Lard, Laurel berry, Laurel leaf oil, Lavender (Lavandula spp.), Lavender oil, Lemon, Lemon balm (Melissa officinalis), Lemon extract, Lemon juice, Lemon Myrtle (Backhousia citriodora), Lemon oil, Lemon verbena (Lippia citriodora), Lemongrass, Lemongrass Oil, Licorice, Long pepper, Lovage, Luohanguo, Lysine, Macadamia oil, Mace, Magnesium, Mahlab, Malabathrum, Maltodextrin, Maltose, Mandarin oil-leavening agent, Manganese, Margarine, Marjoram, Mastic, Meadowfoam seed oil, Mega-purple, Mentha arvensis oil/Mint oil, Methionine, Methyl butyrate, Methyl disulfide, Methyl hexenoate, Methyl isobutyrate, Methyltheobromine, Milk, Milk thistle (Silybum), Mint, Modified starch, Molasses extract, Molybdenum, Mullein, Mustard, Mustard oil (essential oil), Mustard oil (pressed), Mustard plant, Mustard seed, Nigella (Kolanji, Black caraway), Nutmeg, Okra oil, Oleomargarine, Olive oil, Orange oil, Oregano, Oregano oil, Orris root, Palm oil, Panax ginseng, Panax quinquefolius, Pandan leaf, Pantothenic acid (Vitamin B5), Papain, Paprika, Paprika extract, Paprika red, Parsley, Peanut oil/Ground nut oil, Pecan oil, Pectin, Pepper (black, white, and green), Perilla seed oil, Pine needle oil, Pine seed oil, Pistachio oil, Poly vinyl pyrrolidone, Pomegranate seeds, Ponch phoran, Poppy seed, Poppyseed oil, Potassium gluconate, Primrose, Prune kernel oil, Pulegone, Pumpkin seed oil, Purslane, Pyridoxine hydrochloride (Vitamin B6), Quatre épices, Quinoa oil, Ramtil oil, Ras-el hanout, Raspberry, Rice bran oil, Rocket (Arugula), Rosemary, Safflower, Safflower oil, Sage, Saigon Cinnamon, Salad Burnet, Salt, Savory, Sesame oil, Sesame seed, Sodium gluconate, Sorbol, Sorrel, Soybean oil, Spearmint oil, Star anise, Star anise oil, Sugar, Sumac, Sunflower oil, Sweet basil, Sweet cicely, Sweet woodruff, Szechuan pepper (Xanthoxylum piperitum), Tamarind, Tanacetum balsamita, Tandoori masala, Tansy, Tarragon (Artemisia dracunculus), Theine, Thiamine (Vitamin B1), Thyme, Tocopherol (Vitamin E), Trimethylxanthine, Vanilla, Vinegar, Vitamin, Vitamin A (Retinol), Vitamin B1 (Thiamine), Vitamin B12 (Cyanocobalamin), Vitamin B2 (Riboflavin), Vitamin B5 (Pantothenic acid), Vitamin B6 (Pyrodoxine), Vitamin C (Ascorbic acid), Vitamin D (Calciferol), Vitamin E (Tocopherol), Vitamin K (Potassium), Wasabi, Water, Wattleseed, Wheat germ oil and Yucca extract.

### Vehicles

As used herein, the term “vehicle” refers to any substance combined with an active ingredient (e.g. sialic acid, a functional agent or nutraceutical) to aid in the administration and/or application such active ingredient. In some embodiments, a vehicle is a liquid capable of dissolving an active ingredient allowing for dispersion upon application.

### Vehicles: Fats, Oils and Lipid

In some embodiments, nutraceuticals of the present invention may be formulated using one or more of fats, oils or lipids. As used herein, the term “lipid” refers to a small molecule that is hydrophobic and/or amphiphilic including, but not limited to metabolites comprising fats, fatty acids and/or sterols (including but not limited to cholesterol). Depending on their chemical composition, lipids may exist as liquids or solids at ambient temperatures. Triglycerides (triesters of glycerol and fatty acids) are referred to herein as “fat”. Such fats may be either solid or liquid at ambient temperature depending upon the degree of saturation (hydrogenation)/unsaturation of the fatty acid components. Unsaturated fats that are viscous or liquid at ambient temperature are referred to generally as oils. As used herein, the term “oil” refers to any neutrally charged substance, insoluble in water and fluid at ambient temperature. Although insoluble in water, oils typically disperse in alcohols or ethers. Oils are rich in carbon and hydrogen atoms and typically highly combustible. Their neutral charge makes them nonpolar and slippery in consistency. In some embodiments, fats are organic, being derived from natural sources such as plants, animals and/or other organisms capable of metabolic fat synthesis.

In some embodiments, oils that may be suitable vehicles for nutraceutical administration may include, but are not limited to, alpha-linoleic acid, almond oil, apricot kernel oil, avocado oil, babassu oil, bergamot oil, black current seed oil, borage oil, cade oil, camomile oil, canola oil, caraway oil, carnauba oil, castor oil, cinnamon oil, cocoa butter oil, coconut oil, cod liver oil, coffee oil, corn oil, cotton seed oil, emu oil, eucalyptus oil, evening primrose oil, fennel oil, fish oil [including, but not limited to krill oil, sardine oil, herring oil, mackerel oil, eicosapentaenoic acid (EPA), docosahexaenoic acid (DHA), omega-3 fatty acids and N-3 fatty acids], flaxseed oil, geraniol oil, gourd oil, grape seed oil, hazel nut oil, hyssop oil, isopropyl myristate, jojoba oil, kukui nut oil, lavandin oil, lavender oil, lemon oil, litsea cubeba oil, macademia nut oil, mallow oil, mango seed oil, meadowfoam seed oil, mink oil, nutmeg oil, olive oil, orange oil, orange roughy oil, palm oil, palm kernel oil, peach kernel oil, peanut oil, pomegranate seed oil oil, poppy seed oil, pumpkin seed oil, rapeseed oil, rice bran oil, rosemary oil, rose essential oil (including, but not limited to Rosa damascene oil, Rosa alba oil, Rosa gallica oil, Rosa centifolia oil and rose oil), safflower oil, sandalwood oil, sasquana oil, savoury oil, sea buckthorn oil, sesame oil, shea butter oil, silicone oil, soybean oil, sunflower oil, tea tree oil, thistle oil, tsubaki oil, vetiver oil, walnut oil, wheat germ oils, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

In some embodiments, formulation of nutraceuticals with oil increases gastric (e.g. stomach and/or intestine) and plasma retention of functional agents after ingestion. While not limiting, in some embodiments, compositions of the present invention may comprise from about 0.01 to about 0.05, from about 0.025 to about 0.075, from about 0.05 to about 0.2, from about 0.1 to about 0.5, from about 0.25 to about 0.75, from about 0.5 to about 2, from about 1 to about 5, from about 1 to about 10, from about 1 to about 15, from about 2 to about 8, from about 5 to about 10, from about 7.5 to about 15, from about 10 to about 20, from about 15 to about 30, from about 25 to about 50, from about 30 to about 60, from about 50 to about 100, from about 0.1 to about 100 or at least 100 ml of lipid per kg of body weight of the subject ingesting such compositions. In some embodiments, compositions of the present invention are formulated with at least 4, at least 6, at least 8, at least 10, at least 12, at least 14, at least 16, at least 18 or at least 20 ml per kg of body weight of the subject of administration. In some embodiments, compositions of the present invention comprising lipids, may comprise sialic acid-glycoproteins at concentrations (g sialic acid-glycoproteins/100 ml of lipid) of from about 0.01% to about 0.05%, from about 0.02% to about 0.1%, from about 0.5% to about 10%, from about 0.5% to about 2%, from about 1% to about 5%, from about 1% to about 10%, from about 5% to about 10%, from about 5% to about 20%, from about 10% to about 50%, from about 10% to about 25%, from about 20% to about 40%, from about 20% to about 80%, from about 50% to about 75% or at least 75%.

In some embodiments, nutraceutical compositions of the present invention comprising one or more lipid vehicle or excipient to modulate the level of sialic acid in subjects after oral administration. In some embodiments, the level of sialic acid in subjects or in subject tissues and/or fluids is modulated from about 1 to about 12 fold, from about 2 to about 15 fold, from about 3 to about 20 fold, from about 4 to about 25 fold, from about 5 to about 50 fold, from about 10 to about 100 fold, from about 75 to about 200 fold, from about 150 to about 300 fold, from about 250 to about 500 fold, from about 400 to about 1000 fold, from about 750 to about 2000 fold and/or from about 1500 to about 5000 fold.

In some embodiments, delivery vehicles may comprise long chain omega-3 polyunsaturated fatty acids (LC n-3 PUFAs.) As used herein, the term “LC n-3 PUFA” refers to a fatty acid comprising a chain of 20 or more carbon atoms, wherein a double bond exists after the third carbon from the methyl end. LC n-3 PUFAs include, but are not limited to EPA, DHA, hexadecatrienoic acid, alpha-linoleic acid, stearidonic acid, eicosatrienoic acid, eicosapentaenoic acid, heneicosapentaenoic acid, docosapentaenoic acid, clupanodonic acid, tetracosapentaenoic acid and tetracosahexaenoic acid. Consumption of some LC n-3 PUFAs is recommended by health authorities to avoid or manage chronic disease (Russell et al., 2012.) Individual LC n-3 PUFAs or combinations (e.g. EPA and DHA) may be consumed as part of different treatment regimens. Such treatment regimens may comprise up to 0.01 g/day, from about 0.01 to about 0.1 g/day, from about 0.05 to about 0.2 g/day, from about 0.1 to about 0.5 g/day, from about 0.25 to about 1.5 g/day, from about 1 to about 5 g/day, from about 2.5 to about 7.5 g/day, from about 5 to about 10 g/day or at least 10 g/day. In some cases, the incorporation of one or more LC n-3 PUFAs into delivery vehicles of the present invention may be carried out as part of a combined treatment for increased sialic acid uptake and treatment of one or more diseases, disorders and/or conditions where LC n-3 PUFAs may be therapeutic. Such diseases, disorders and/or conditions may include, but are not limited to cardiovascular diseases, disorders and/or conditions, rheumatoid arthritis, elevated blood pressure and/or mental diseases, disorders and/or conditions.

### Vehicles: Liposomes, Lipoplexes and Lipid Nanoparticles

Nutraceuticals of the present invention may be formulated using one or more liposomes, lipoplexes, or lipid nanoparticles. In some embodiments, nutraceutical compositions may further comprise liposomes. Liposomes are artificially-prepared vesicles which may primarily comprise one or more lipid bilayers and may be used as a delivery vehicle for the administration of compounds of the present invention. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the nutraceutical formulations.

The formation of liposomes may depend on the physicochemical characteristics such as, but not limited to, the nutraceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, formulations may be designed or compositions altered such that they passively or actively are directed to different cell types in vivo.

Formulations can also be selectively targeted through expression of different ligands on their surface as exemplified by, but not limited by, folate, transferrin, N-acetylgalactosamine (GalNAc), and antibody targeted approaches.

Liposomes, lipoplexes, or lipid nanoparticles may be used to improve the efficacy of nutraceutical function as these formulations may be able to increase cell transfection with nutraceuticals. The liposomes, lipoplexes, or lipid nanoparticles may also be used to increase the stability of nutraceuticals.

### Peptide and Protein Formulations

Nutraceuticals of the invention may be formulated with peptides and/or proteins. In formulations of the present invention, peptides or proteins may be incorporated to increase cell transfection and/or alter the biodistribution of nutraceuticals (e.g., by targeting specific tissues or cell types).

### Administration and Delivery

The compositions of the present invention may be administered by any of the standard methods or routes known in the art. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compositions of the invention may be formulated for administration topically. The skin may be an ideal target site for delivery as it is readily accessible. Three routes are commonly considered to deliver nutraceutical compositions to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Compositions may be delivered to the skin by several different approaches known in the art.

In some embodiments, the invention provides for a variety of dressings (e.g., wound dressings) or bandages (e.g., adhesive bandages) for conveniently and/or effectively carrying out methods of the present invention. Typically dressing or bandages may comprise sufficient amounts of nutraceutical compositions described herein to allow a user to perform multiple treatments of a subject(s).

Dosage forms for topical and/or transdermal administration of compositions may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing compounds in the proper medium. Alternatively or additionally, rate may be controlled by either providing a rate controlling membrane and/or by dispersing compounds in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, compositions of the present invention are formulated in depots for extended release. Generally, a specific organ or tissue (a “target tissue”) is targeted for administration.

In some aspects of the invention, functional agents are spatially retained within or proximal to a target tissue. Provided are methods of providing compositions to one or more target tissue of a subject by contacting the one or more target tissue (comprising one or more target cells) with compositions under conditions such that the functional agents are substantially retained in the target tissue, meaning that at least 10, 20, 30, 40, 50, 60, 70, 80, 85, 90, 95, 96, 97, 98, 99, 99.9, 99.99 or greater than 99.99% of the functional agents are retained in the target tissue. Advantageously, retention is determined by measuring the level of functional agents present in the compositions entering the target tissues and/or cells. For example, at least 1, 5, 10, 20, 30, 40, 50, 60, 70, 80, 85, 90, 95, 96, 97, 98, 99, 99.9, 99.99 or greater than 99.99% of functional agents administered to the subject are present intracellularly at a period of time following administration. For example, intramuscular injection to a subject is performed using an aqueous composition comprising one or more functional agents and a transfection reagent, and retention of the functional agents is determined by measuring the level of functional agents present in the muscle cells.

Certain aspects of the invention are directed to methods of providing compositions to target tissues of subjects, by contacting the target tissues (containing one or more target cells) with compositions under conditions such that the compositions are substantially retained in the target tissue. Compositions contain an effective amount of functional agents such that the effect of interest is produced in at least one target cell. Compositions generally contain cell penetration agents and a pharmaceutically acceptable carrier, although “naked” functional agents (such as functional agents without cell penetration agents or other agents) are also contemplated.

### Pulmonary Administration

Pharmaceutical compositions may be prepared, packaged, and/or sold in formulations suitable for pulmonary administration via the buccal cavity. Such formulations may comprise dry particles further comprising functional agents and having a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Nutraceutical compositions formulated for pulmonary delivery may provide functional agents in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of nutraceutical compositions. Another formulation suitable for intranasal administration is a coarse powder comprising functional agents and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. Nutraceutical compositions may be prepared, packaged, and/or sold in a formulation suitable for buccal administration. Such formulations may, for example, be in the form of tablets and/or lozenges made using conventional methods, and may, for example, comprise 0.1% to 20% (w/w) functional agents, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising functional agents. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic or Otic Administration

Nutraceutical compositions may be prepared, packaged, and/or sold in a formulation suitable for ophthalmic or otic administration. Such formulations may, for example, be in the form of eye or ear drops including, for example, a 0.1/1.0% (w/w) solution and/or suspension of functional agents in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Subretinal inserts may also be used as a form of administration.

### Fasting Administration

In some embodiments, nutraceutical compositions of the present invention are administered to fasting subjects. As used herein, the term “fasting subject” refers to a subject that has not ingested solids or liquids (with the exception of water) by way of the gastrointestinal tract for a period time, typically at least 1 hour. In some embodiments, such periods of time may be at least 2 hours, at least 3 hours, at least 4 hours, at least 5 hours, at least 6 hours, at least 7 hours, at least 8 hours, at least 9 hours, at least 10 hours, at least 12 hours, at least 24 hours and/or at least 48 hours.

### Combinations

Nutraceutical compositions may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each composition will be administered at a dose and/or on a time schedule determined for that composition. In some embodiments, the present invention provides for the delivery of compositions in combination with agents that may improve their bioavailability, reduce and/or modify their metabolism, inhibit their excretion, and/or modify their distribution within the body.

### Dosage

The present disclosure encompasses delivery of nutraceutical compositions for any of therapeutic, pharmaceutical, diagnostic or imaging purpose by any appropriate route taking into consideration likely advances in the sciences of drug delivery. Delivery may be naked or formulated.

### Naked Delivery

Functional agents of the present invention may be delivered to cells, tissues, organs or organisms in naked form. As used herein in, the term “naked” refers to functional agents delivered free from agents or modifications which promote transfection or permeability. Naked functional agents may be delivered to cells, tissues, organs and/or organisms using routes of administration known in the art and described herein. Naked delivery may include formulation in a simple buffer such as saline or PBS.

### Formulated Delivery

Nutraceutical compositions of the present invention may be formulated, using methods described herein. Formulations may comprise functional agents which may be modified and/or unmodified. Formulations may further include, but are not limited to, cell penetration agents, pharmaceutically acceptable carriers, delivery agents, bioerodible or biocompatible polymers, solvents, and sustained-release delivery depots. Formulated nutraceutical compositions may be delivered to cells using routes of administration known in the art and described herein.

Compositions may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Dosing

The present invention provides methods comprising administering one or more nutraceutical compositions in accordance with the invention to a subject in need thereof. Nucleic acids encoding components of nutraceutical compositions, proteins or complexes comprising functional agents, or pharmaceutical, imaging, diagnostic, or prophylactic compositions thereof, may be administered to a subject using any amount and any route of administration effective for preventing, treating, diagnosing, or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

According to the present invention, nutraceutical compositions may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in a 24 hr period. It may be administered as a single unit dose. In some embodiments, nutraceutical compositions of the present invention are administered to a subject in split doses. Nutraceuticals may be formulated in buffer only or in a formulation described herein. Nutraceutical compositions comprising functional agents as described herein may be formulated into a dosage form described herein, such as a topical, intranasal, intratracheal, or injectable (e.g., intravenous, intraocular, intravitreal, intramuscular, intracardiac, intraperitoneal or subcutaneous). General considerations in the formulation and/or manufacture of functional agents may be found, for example, in Remington: The Science and Practice of Pharmacy 21^{st} ed., Lippincott Williams & Wilkins, 2005 (incorporated herein by reference).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which may be used include polymeric substances and waxes. Solid compositions of a similar type may be employed as fillers in soft and hard-filled gelatin capsules using such excipients as lactose or milk sugar as well as high molecular weight polyethylene glycols and the like.

### DEFINITIONS

Adjacent: As used herein, the term “adjacent” refers to something that is adjoining, neighboring or next to a given entity. In some embodiments, “adjacent residues” are sugar residues within a glycan chain that are linked to one another. In some embodiments, “adjacent glycans” are glycan chains that next to each other either in direct contact or within close proximity and without another glycan in between the two.

Administered in combination: As used herein, the term “administered in combination” or “combined administration” means that a subject is simultaneously exposed to two or more agents administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more nutraceutical compositions, as described herein, are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biomolecule: As used herein, the term “biomolecule” is any natural molecule which is amino acid-based, nucleic acid-based, carbohydrate-based or lipid-based, and the like.

Branch: As used herein, the term “branch” refers to an entity, moiety or appendage that is linked or extends out from a main entity or source. In some embodiments, a “branch chain” or “branching chain” is a polysaccharide chain that extends from a parent chain. As used herein, a “parent chain” is used to refer to the polysaccharide from which a branching chain is linked. In the case of a glycan with multiple branches, the parent chain may also refer to the source chain from which all such branches are directly or indirectly attached. In the case of a polysaccharide comprising a chain of hexose residues, parent chain linkages typically occur between carbons 1 and 4 of adjacent residues while branching chains are attached to a parent chain through a linkage between carbon 1 of the branching residue and carbon 3 of the parent residue from which the branch extends. As used herein, the term “branching residue” refers to the residue attached to the parent chain in a branching chain.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Cyclic or Cyclized: As used herein, the term “cyclic” refers to the presence of a continuous loop. Cyclic molecules need not be circular, only joined to form an unbroken chain of subunits.

Cytidine monphosphate-N-acetylneuraminic acid hydroxylase: As used herein, the term “cytidine monophosphate-N-acetylneuraminic acid hydroxylase” or “CMAH” refers to an enzyme, absent in humans, but present in most other mammals (including, but not limited to mice, pigs and chimpanzees) that catalyzes the formation of N-glycolylneuraminic acid from N-acetylneuraminic acid. The absence of the enzyme in humans is due to a frameshift mutation resulting in the premature termination of the CMAH transcript and the production of a non-functional protein.

Cytotoxic: As used herein, the term “cytotoxic” is used to refer to an agent that kills or causes injurious, toxic, or deadly effects on a cell [e.g., a mammalian cell (e.g., a human cell)], bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of an agent.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Distal: As used herein, the term “distal” means situated away from the center or away from a point or region of interest.

Edible: As used herein, the term “edible” refers to a substance that may be ingested by a subject by way of the gastrointestinal tract without significantly harmful effects (e.g. erosion of tissue, vomiting, etc). In some embodiments, compounds and compositions of the present invention are edible.

Engineered: As used herein, embodiments of the invention are “engineered” when they are designed to have a feature or property, whether structural or chemical, that varies from a starting point, wild type or native molecule. Thus, engineered agents or entities are those whose design and/or production include an act of the hand of man.

Ether bond: As used herein, an “ether bond” refers to a chemical bond comprising an oxygen bonded between two carbon atoms. In some embodiments, ether bonds link sugar residues to one another in a glycan chain. Such bonds are also referred to as “glycosidic bonds” or “glycosidic linkages”. In some embodiments, ether bonds link glycans to protein, typically forming a link between a sugar residue and an amino acid residue. Such amino acid residues include serine and threonine. In some embodiments, ether bonds link glycans to a glycan array comprising a carbohydrate linker that participates in bond formation.

Expression: As used herein, “expression” of a protein refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least one functional agent and at least one delivery agent.

Functional agent: As used herein, a “functional agent” is an entity which exhibits or promotes a property and/or activity by which it is characterized.

Glycan: As used herein, the terms “glycan”, “oligosaccharide” and “polysaccharide” are used interchangeably and refer to polymers made up of sugar monomers, typically joined by glycosidic bonds also referred to herein as linkages. In some embodiments, the terms “glycan”, “oligosaccharide” and “polysaccharide” may be used to refer to the carbohydrate portion of a glycoconjugate (e.g., glycoprotein, glycolipid or proteoglycan).

Glycan chain: As used herein, the term “glycan chain” refers to a sugar polymer comprising two or more sugars. In some embodiments, glycan chains are covalently linked to proteins through serine or threonine residues on the protein.

Glycan-rich composition: As used herein, the term “glycan-rich composition” refers to composition comprising a large percentage of glycans. In some embodiments, glycans within a glycan-rich composition may comprise from about 1% to about 10%, from about 5% to about 15%, from about 20% to about 40%, from about 30% to about 50%, from about 60% to about 80%, from about 70% to about 90% or at least 100% of the total weight of the composition.

Glycosidic bond: As used herein, the term “glycosidic bond” refers to a covalent bond formed between a carbohydrate and another chemical group. In some embodiments, glycosidic bonds are formed between the reducing end of one sugar molecule and the non-reducing end of a second sugar molecule or polysaccharide chain. Such glycosidic bonds are also known as 0-glycosidic bonds due to the oxygen (or ether bond) between the joined sugars. In some embodiments, a glycosidic bond between two sugars or between a sugar and a linker may also be referred to as a “linkage”.

Inflammation: As used herein, the term “inflammation” refers to a complex immunological response in biological organisms, typically triggered by immunogenic stimuli, such as pathogens or foreign agents. In some embodiments, immunogenic stimuli may comprise self- or auto-antigens causing auto-immune inflammation. As used herein, the term “anti-inflammatory” refers to an agent capable of preventing, reducing or eliminating inflammation in a subject.

Inflammatory biomarker: As used herein, the term “inflammatory biomarker” refers to a chemical or protein that is indicative of increased or decreased inflammation in a subject.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Knockout: As used herein, the term “knockout” refers to an organism wherein an existing gene has been inactivated through a process that typically involves the hand of man. In a knockout organism, a gene that has been inactivated is said to have been “knocked out”. In some embodiments, the knocked out gene may be inactivated through the insertion of a nucleotide sequence into the gene or through replacement of the gene entirely.

Linker: As used herein, a “linker” refers to a moiety that connects two or more domains, moieties or entities. In one embodiment, a linker may comprise 10, 11, 12, 13, 14, 15 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent) or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris(2-carboxyethyl)phosphine (TCEP), or other reducing agents, and/or photolysis, as well as an ester bond which may be cleaved for example by acidic or basic hydrolysis. In some embodiments, a linker is a carbohydrate moiety used to link glycans to a substrate, such as in a glycan array. Such carbohydrate linkers include, but are not limited to —O(CH₂)₂CH₂HN₂ and —O(CH₂)₃NHCOCH₂ (OCH₂CH₂)₆NH₂.

Mucin: As used herein, the term “mucin” refers to a family of proteins that are heavily glycosylated. In some embodiments mucins are produced by the submaxillary glands and are found in saliva and mucous.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Peptide: As used herein, a “peptide” is a chain of amino acids joined by peptide bonds that is less than or equal to 50 amino acids in length, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than active agents (e.g., as described herein) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proximal: As used herein, the term “proximal” means situated nearer to the center or to a point or region of interest.

Residue: As used herein, the term “residue” refers to a monomer associated with or capable of associating with a polymer. In some embodiments, residues comprise sugar molecules including, but not limited to glucose, galactose, N-acetylglucosamine, N-acetylgalactosamine and sialic acids.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Sialyl: As used herein, the prefix “sialyl” as well as the term “sialylated” describe compounds comprising sialic acid.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc).

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Submaxillary glands: As used herein, the term “submaxillary glands” or “submandibular glands” refer to mucous producing glands located beneath the mouth floor. These glands are capable of producing mucins and in some embodiments, may be extracted from mammals as a source of mucin.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of nutraceutical compositions of the present invention may include chemical and/or enzymatic methods.

Therapeutic agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., functional agent, nutraceutical, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transgenic: As used herein, the term “transgenic” refers to an organism that comprises one or more genes incorporated within the organisms genome that are not naturally found in that organism.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Wild type: As used herein, the term “wild type” refers to an organism comprising a natural genome (free from genes derived from other organisms).

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1

### Preparation of Red Blood Cell (RBC) Ghosts

### Comparison of Sialic Acid Content Between Three Species

In order to select a species from which to prepare RBC ghost samples, RBC ghosts from three sources were analyzed for sialic acid content: mouse, bovine and porcine. 1,2-diamino-4,5-methyleneoxybenzene (DMB) high pressure liquid chromatography (HPLC) was performed to analyze sialic acid type and to quantify sialic acid (especially Neu5Gc) content in each sample.

30 ml of blood from each source was centrifuged at 2000× gravity for 10 minutes at 4° C. to remove blood plasma and buffy coat. Pellets were washed with ice cold phosphate buffered saline (PBS) before centrifugation again at 2,000× gravity for 10 minutes at 4° C. This step was repeated before pellets were subjected to lysis with 150 ml of cold Tris-EDTA (TE) buffer (10 mM Tris, 1 mM EDTA) for 10 minutes on ice with intermittent mixing. Samples were then centrifuged at 10,000× gravity for 20 minutes at 4° C. Supernatants were discarded and pellets were washed 4 times with 150 ml of ice cold TE buffer containing 30 mM NaCl. Pellets were then washed with 150 ml of ice cold 10 mM HEPES buffer (pH 7.5) with 0.5 mM MgCl₂, 0.05 mM CaCl₂ and 50 mM KCl. Pellets were resuspended in 40 ml of TE buffer (without NaCl) and placed in pre-weighed ultracentrifuge tubes in order to evaluate wet weight. Samples were then centrifuged for 20 minutes at 50,000× gravity. Supernatants were discarded and samples were weighed again. The amount of RBC ghost (μg) was determined by subtracting sample wet weight from sample weight after the final centrifugation. Samples were resuspended in 10 ml of TE buffer before further analysis.

Samples were next analyzed by DMB-HPLC to determine total sialic acid content, Neu5Ac content and Neu5Gc content. Table 1 provides the results of the analysis. Total sialic acid (pmol/μg) is the calculated sialic acid content (pmol) in RBC ghosts (μg). It is equal to the Neu5Ac content plus the Neu5Gc content in +acid −base treated samples. The percent of Neu5Gc is calculated by dividing the amount of Neu5Gc detected from the total amount of Neu5Ac plus Neu5Gc. The data provided do not include other modified forms of sialic acid, such as Neu5,7Ac, Neu5,8Ac₂, Neu5,9Ac₂ and Neu5Gc9Ac. Of these modified forms, Neu5,9Ac2 is the major form, making up 0.8% of the total sialic acid. The other modified forms are less than 0.2%.

TABLE 1
Neu5Gc content of RBC ghosts from different sources

| | Total sialic acid (pmol/μg) | % Neu5Gc |
|---|---|---|
| Mouse RBC Ghosts | 0.35 | 4.64 |
| Bovine RBC Ghosts | 2.23 | 91.13 |
| Porcine RBC Ghosts | 0.80 | 95.42 |

300 pmol of Neu5Gc (from a mucin source) was used in an alternate study for immunization of mice, giving a good response. Results of this study are detailed in Table 2.

TABLE 2
Anti-Neu5Gc immune response resulting from mucin Neu5Gc

| | Stock (μg/μl) | μl/300 pmol Neu5Gc |
|---|---|---|
| Mouse RBC Ghosts | 286 | 65.44 |
| Bovine RBC Ghosts | 175 | 0.84 |
| Porcine RBC Ghosts | 351 | 1.12 |

### Example 2

### Porcine Submaxillary (PSM) Mucus Preparation

150 cryoground submaxillary glands (Pelfreeze Biologicals, Rogers, AR) were purchased and kept frozen at −20° C. until use. 700 g of cryoground glands were weighed and added to a 4 L beaker with 3.5 L of purified water. The solution was stirred at 4° C. for 6-8 hours using a magnetic stir bar. Stirring was then halted and the solution was allowed to settle overnight at 4° C. A funnel (10 inch diameter) was prepared by fluffing about 2 inches of glass wool and softly plugging the bottom of the funnel. About 4 inches of glass wool was then fluffed and layed on top of the plug. Next, a mesh (Home Depot, Atlanta, Ga.) with 0.25 mm pores was placed on top. Supernatant from the submaxillary gland solution was collected at 4° C. by slowly filtering through the padded funnel. Solid waste caught in the filter was discarded. The filtered supernatant was acidified to pH 3.5 by slowly adding 40-50 ml of 1 M HCl while stirring. The solution was stirred at 4° C. for 8 hours and then allowed to settle overnight at 4° C. The settled material, containing mucins, was collected by siphoning off the supernatant. Mucins were prepared by spinning at 400×g for 15 min at 4° C. in a Sorvall centrifuge (Thermo Fisher Scientific, Waltham, Mass.). The supernatant was discarded and the pellet was resuspended in purified water and spun again at 400×g for 15 min at 4° C. in a Sorvall centrifuge. The supernatant was again discarded and the pellet was collected by resuspension in a minimal volume of purified water. The pH of the pellet solution was adjusted to pH 8.0 using 1.5 M NaOH before stirring the solution overnight at 4° C. to homogenize the solution. The mucin solution was then dialyzed against distilled water using 140 mm wide dialysis tubing. 0.2 L batches were dialyzed in a large bucket with 5 changes of water. A freezing bath was then prepared by mixing ethanol with dry ice in a styrofoam box. Clean lyophilizer bottles were placed into the freezing bath and dialyzed mucin solution was poured slowly into each bottle to allow for freezing. Lyophilizer bottles containing frozen mucin solution were then transferred to a lyophilizer and dried for about 7 days. Bottle weights were recorded and bottles were stored at −20° C. in vacuum-sealed bags until use.

### Example 3

### Feeding Study

A feeding study is carried out to assess diet-induced sialic acid uptake and incorporation as well as to assess diet-induced anti-Neu5Gc antibody production in Neu5Gc-deficient mice. Male and female cmah−/− mice as well as female wild type controls are maintained on control chow diets or chow diets comprising PSM or GMP-PSM according to the schedule outlined in Table 3.

TABLE 3
Feeding study plan

| | Num. of | | | | | Blood | |
|---|---|---|---|---|---|---|---|
| | mice | | Chow | Chow | | collection | |
| Group | (sex) | Strain | (week 0-8) | (week 9-12) | Immunization | (week #) | Endpoints |
| 1 | 6 (male) | cmah−/− | PSM | | No | 2, 4, 6, 8, | 1. Body |
| 2 | 7 (female) | cmah−/− | PSM | | Yes | 10 and 12 | weight, |
| 3 | 7 (male) | cmah−/− | PSM | GMP-PSM | No | | condition |
| 4 | 7 (female) | cmah−/− | PSM | GMP-PSM | Yes | | 2. Blood |

| | | | | | | |
|---|---|---|---|---|---|---|
| 5 | 3 (female) | cmah−/− | Control | No | 12 | collection |
| 6 | 3 (female) | wild type | Control | No | 12 | 3. Tissue |
| | | | | | | collection |

Mice from groups 2 and 4 are immunized with Freunds Complete adjuvant mixed with immunogen [Neu5Gc from porcine red blood cell (RBC) ghosts], subcutaneously around armpits and inguinal regions (50 μl/site, 4 sites, 200 μl/mouse) on day 14 of the study. These mice are also immunized with Freunds Incomplete adjuvant mixed with immunogen (Neu5Gc from porcine RBC ghosts), subcutaneously around armpits and inguinal regions (50 μl per site, 4 sites, 200 μl/mouse) on days 28, 42 and 56.

Blood is collected according to the schedule provided in Table 3. At each collection, approximately 0.2 ml of whole blood is collected from each animal via facial vein bleed. Blood is collected into serum separator tubes and kept at room temperature for at least 30 minutes to allow clotting. The blood is then processed to serum by centrifugation at 1,500× gravity for 5 minutes and stored at −80° C. until further analysis.

After terminal blood collection, animals are euthanized and subjected to perfusion and tissue collection. Animals are perfused by cardiac perfusion at a rate of about 7 ml/minute. The first perfusion solution comprises 20 ml of warmed Krebs-Ringer solution (125 mM NaCl, 2.5 mM KCl, 1.25 mM NaH₂PO₄, 2 mM CaCl₂, 1 mM MgCl₂, 25 mM NaHCO₃ and 25 mM glucose, pH 7.4) supplemented with 10 mM EDTA. The second perfusion solution comprises 20 ml Krebs-Ringer solution supplemented with 150 μM CaCl2 and 0.5 mg/ml collagenase type I. Following perfusion, liver, aorta, kidneys and small intestine are collected. Each collected tissue is divided and portions of each are prepared for further analysis in one of three ways: 1. frozen in OCT medium (Sakura, Alphen aan den Rijn, The Netherlands) for immunohistochemical (IHC) analysis, 2. subjected to collagenase digestion for fluorescence-activated cell sorting (FACS) analysis or 3. snap frozen in liquid nitrogen for long term storage. Livers are cut into two pieces (left and right). Left halves are processed for IHC while right halves are divided for FACS analysis and storage. For kidneys, each left kidney is processed for IHC while right kidneys are processed for long term storage only. Aortas are cut lengthwise, then cut crosswise. Upper halves are used for IHC while lower halves are processed for FACS analysis and long term storage. Small intestinal samples are cut into 3 sections (duodenum, jejunum and ileum), cut lengthwise to open and finally crosswise to get half for IHC and half to divided for FACS analysis and long term storage.

### Analysis of Tissue Samples

Tissue samples are assessed for Neu5Gc levels.

### Example 4

### Analysis of Serum Samples for Inflammatory Biomarkers

Serum samples collected according to Example 3 from Group 2 (maintained on a diet comprising Neu5Gc) and Group 4 (Neu5Ac was introduced into their diet at day week 9) mice were analyzed for levels of inflammatory biomarkers including INFγ, IL-10, IL-12p70, IL-13, IL-17A, IL-1β, IL-2, IL-4, IL-5, IL-6, KC, MCP-1, TNFα, Haptoglobin and SAA. Levels were assessed using samples collected at week 8 and week 13 of the study.

Sera were screened by enzyme-linked immunoassay (ELISA) to detect biomarker levels. Individual wells of each ELISA plate were coated with primary antibodies, such that each well comprised antibodies directed toward only one specific inflammatory biomarker. Coated plates were then blocked and incubated with serum samples. Plates were then rinsed and incubated with secondary antibodies conjugated with a detectable label for one hour. Plates were rinsed again, treated with HRP substrate and examined spectrophotometrically for absorbance at 490 nm.

ELISA results were used to determine the percent increase in inflammatory biomarker levels in serum samples between weeks 8 and 13 of the study (Table 4). Remarkably, percent increases in many inflammatory biomarker levels were higher in Group 2 mice as compared to Group 4 mice over the same period.

TABLE 4
% change in levels between weeks 8 and 13 of the study

| Biomarker | Group 2 | Group 4 |
|---|---|---|
| INFγ | 25% | −8% |
| IL-10 | 30% | 3% |
| IL-12p70 | 4% | 24% |
| IL-13 | 17% | 17% |
| IL-17A | −20% | −32% |
| IL-1β | 83% | 82% |
| IL-2 | 49% | 53% |
| IL-4 | 65% | 14% |
| IL-5 | 40% | 16% |
| IL-6 | 34% | −14% |
| KC | 65% | 26% |
| MCP-1 | 24% | 2% |
| TNFα | 42% | −21% |
| Haptoglobin | −79% | −115% |
| SAA | −21% | −23% |

### Example 5

### Analysis of Samples by Glycan Microarray

Analyses of samples by glycan microarray are carried out essentially as described in international application number PCT/US2013/029240 filed on Mar. 6, 2013, the contents of which are herein incorporated by reference in their entirety. Glycan microarray slides are scanned prior to and after ethanolamine blocking to look for changes in background signal. Ethanolamine blocking is carried out by first preparing a blocking solution comprising 0.1 M Tris, 0.05 M ethanolamine and a pH of 9.0. The solution, as well as a 1.5 L beaker of double-distilled water (for washes) are heated to 50° C. The slides are arranged in the slide holder and quickly submerged in staining tubs with blocking buffer at 50° C. (slides are shaken lightly to dislodge bubbles from the slides). Staining tubs are placed on a shaker for 60 minutes. Washing tubs are prepared with pre-warmed (50° C.) double-distilled water. Slides are rinsed by submersion in the first tub of water with gentle shaking and then transferred to the second tub where they are placed on a shaker for 10 minutes or more. 2 additional washing tubs are prepared and slides are transferred to the first and then to the second tub for 1 minute. Slides are then removed and dried by centrifugation at 200×g for 5 min and stored in an airtight bag in the dark until use.

For slide development, an appropriate volume of blocking and washing solutions are prepared. Blocking solution is prepared by combining phosphate buffered saline (PBS) with ovalbumin (at 1% of final volume). Washing solution 1 is PBST [PBS (pH 7.3) with 0.1% Tween-20]. Washing solution 2 is PBS (pH 7.3). Washing solution 3 is double-distilled water.

Next, each slide is placed into the Arraylt hybridization tool (Arrayit corporation, Sunnyvale, Calif.). Each microarray well is filled with 200 μl of blocking solution and incubated for 1 hour in a humid chamber at room temperature with gentle shaking before dumping out the solution. Group 1 mouse (as described in example 3) serum samples are prepared in blocking solution at a ratio of 1:250 and added to individual microarray wells (200 μl/well). Wells are incubated for 1 hour at room temperature in a humid chamber with shaking 200 μl of PBS is next added to each well, slides are shaken for 1 min and then emptied. Slides are then immediately washed with washing solution 1, quickly emptied and filled again with washing solution 1 before shaking for 10 minutes at room temperature. Washing solution 1 is discarded from the wells, wells are refilled with washing solution 2 and then emptied. Remaining washing solution in wells is aspirated before proceeding to the next step.

For detection of bound antibodies, 200 μl of secondary antibody solution [Cy3-conjugated anti-mouse IgG antibody (Jackson Laboratories, Bar Harbor, Me.) diluted 1:500 in blocking solution] is added to each well and incubated for 1 hour in the dark at room temperature in a humid chamber with shaking 200 μl of PBS is then added, the wells are shaken for an additional minute and wells are emptied. Wells are then washed quickly with washing solution 1, emptied and washed again with washing solution 1 for 10 minutes with shaking Wells are then emptied and rinsed in a tub of PBS followed by rinsing in a tub of double-distilled water.

Additional washes are carried out by submerging slides in a tub of PBS for 10 minutes at room temperature with shaking, followed by a quick submersion in a tub of double-distilled water and 10 minute incubation in a tub of double-distilled water at room temperature with shaking After washing, slides are spun at 200× gravity for at least 5 minutes to remove all excess water and avoid water stains.

Slides are scanned using GenePix 4000b (Molecular Devices, Sunnyvale, Calif.) immediately for fluorescent signal at 532 nm and 635 nm wavelength under 100% laser power, a 350 gain setting and 10 μm pixel resolution [laser set using GenPixPro 7 software (Molecular Devices, Sunnyvale, Calif.)]. Intensity values are calculated by subtracting raw fluorescence (532 nm) intensity values for each replicate (4 replicates in total) from background fluorescence (532 nm) levels, then averaging the resulting replicate groups to generate an intensity value for each replicate group.

### Example 6

### ELISA Analysis for the Identification of Immune-Responsive Mice

ELISA analyses are carried out essentially as described in international application number PCT/US2013/029240 filed on Mar. 6, 2013, the contents of which are herein incorporated by reference in their entirety. Sera are screened by enzyme-linked immunoassay (ELISA) to identify mice immune responsive to PSM vaccinations. Screening is carried out using de-O-acetylated bovine submaxillary mucin (BSM) as a target. BSM is chosen due to the presence of an antigenically different protein core from that of PSM. This prevents protein-specific antibodies from interfering with the assay. The glycan portion of BSM is similar to that of PSM with the exception of increased levels of 9-O-acetylated sialic acid.

ELISA plates are coated with BSM followed by base treatment to destroy 9-O-acetylation of BSM. Plates are blocked and incubated with serum samples to look for binding. Samples are treated with or without perioidate, a chemical that destroys the C6 side chain of sialic acids. This is done to reveal whether or not binding in untreated samples is sialic acid-specific. As another control, samples are incubated with or without 20 mM Neu5Ac or Neu5Gc to look for the ability of these sialic acids to compete for binding of sera components. Plates are then rinsed and incubated with secondary antibody (anti-mouse IgG-HRP, Jackson Immunoresearch Laboratories, West Grove, Pa.) for one hour. Plates are rinsed again, treated with HRP substrate and examined spectrophotometrically for absorbance at 490 nm.

Specificity of the mouse sera for Neu5Gc over Neu5Ac is determined through analysis of ELISA results from samples that are subjected to competition with free Neu5Ac.

### Example 7

### Analysis of Commercially Available GMP for Sialic Acid Content

GMP from three vendors was analyzed for sialic acid content. DMB-HPLC was utilized in order to analyze individual sialic acid types and to quantify sialic acid content in each (especially Neu5Ac). Samples were received from Davisco (Eden Prairie, Minn.), Farbest Foods (Huntingburg, Ind.) and Murray Goulburn (Brunswick, Australia). The results of the analysis are presented in Table 5.

TABLE 5
Sialic acid content in commercially available foods

| | % sialic acid of total GMP | % Neu5Ac |
|---|---|---|
| Davisco | 8.44 | 99.23 |
| Farbest Foods | 4.61 | 99.05 |
| Murray Goulburn | 6.05 | 98.93 |

The percent sialic acid is calculated as the percent of total sialic acid (Neu5Ac+Neu5Gc) in GMP. The percent Neu5Ac is determined by dividing the amount of Neu5Ac by the total amount of sialic acid present (Neu5Ac+Neu5Gc). These calculations do not include other modified forms of sialic acid such as Neu5,7Ac₂, Neu5,8Ac₂, Neu5,9Ac₂ and Neu5Gc9Ac. Besides Neu5Ac and Neu5Gc, Neu5,9Ac₂ is the major type of modified sialic acid present (up to 0.8%). Other modified forms represent less than 0.2%.

### Example 8

### Isolation of Glycans and Glycopeptides Comprising Sialic Acid from Egg Components

Glycans and glycopeptides comprising sialic acids are obtained according to the methods disclosed by Seko et al (Seko, A. et al., Biochimica et Biophysica Acta. 1997. 1335(1-2):23-32.) 1.9 liters of egg yolks are obtained from chicken eggs collected within a half day of being laid. All isolation steps are carried out at 4° C. Unfertilized eggs are combined 1:1 with water and mixed with 1/10 volume of phenol/water (9:1, w/w), stirring vigorously for 2 hours. The resulting emulsion is combined with 4.8 liters of water and centrifuged at 6,000 rpm for 30 minutes. Resulting supernatant is applied to a Sephadex G-50 gel filtration column (Sigma-Aldrich, St. Louis, Mo.) and eluted with 0.1 M NaCl. The elution is monitored using the resorcinol-HCl method (for elution of sialic acid) and the phenol-H2SO4 method (for elution of hexoses) (Svennerholm, L. Biochim. Biophys. Acta, 1957. 24:604-11; Dubois, M. et al., Anal. Chem. 1956. 28:350-6.) Fractions comprising sialic acid are collected and rechromatographed on the same column. Fractions are then desalted using a Sephadex G-25 column (Sigma-Aldrich, St. Louis, Mo.) eluted with 5% ethanol. Effluent is applied to an anion exchange column and equilibrated with 5 mM Tris-HCl buffer, pH 8.0. Substances adsorbed are next eluted by linear salt gradient, also in 5 mM Tris-HCl buffer, pH 8.0. Sialylglycopeptides are present in resulting flow through solutions, while free sialylglycans are eluted by NaCl linear gradient elution from the column. Both fractions are purified further using cation exchange chromatography before salt removal and lyophilization. Sugar compositions are further analyzed using gas-liquid chromatography.

### Example 9

### Immunohistochemical Analysis of Feeding Study Subject Tissues

Tissues from mice subjected to the feeding study according to Example 3 were analyzed by immunohistochemical staining. At the termination of the feeding study, mouse tissues were processed for frozen sectioning. Frozen sections from trachea, lungs, liver, pancreas, skin and small intestines were placed on glass slides and subjected to immunohistochemical analysis. Both untreated as well as periodate-treated sections were used for analysis. Glass slides with sections were air dried overnight at room temperature. The next day, sections were rehydrated in phosphate buffered saline (PBS) and blocked for 20 minutes with 0.3% hydrogen peroxide in PBS. Next, slides were treated with PBS with 0.5% fish gelatin. Biotin/avidin blocking was carried out using a kit (Vector Laboratories, Burlingame, Calif.), product number SP2001. First sections were treated with avidin blocking solution for 15 minutes. Then slides were washed with PBS with 0.05% TWEEN® 20 (PBST) and treated again with PBS with 0.5% fish gelatin. Next, slides were treated for 15 minutes with biotin blocking solution before being washed with PBST. Slides were then subjected to fixation with 10% neutral buffered formalin for 30 minutes. This was followed by washing with PBST and re-treatment with PBS with 0.5% fish gelatin for 10 minutes.

Sections were next treated with primary antibody or treated with chicken IgY as a negative control. Sections treated with primary antibody were treated with either Chicken anti-Ne5Gc or Chicken anti-Neu5Gc in 20% Chimp Serum. Primary antibodies were diluted 1:200 in PBS with 0.5% fish gelatin and incubated with sections for 1 hour at room temperature, followed by overnight at 4° C. The next day, slides were washed with PBST before being treated with secondary antibody, biotinylated donkey anti-chicken, 1:500 in PBS with 0.5% fish gelatin for 30 minutes at room temperature. Sections were once again washed with PBST before being treated with horseradish peroxidase-coated streptavidin particles at a dilution of 1:500 in PBS with 0.5% fish gelatin for 30 minutes at room temperature. After a final wash, slides were incubated with a chromagen solution (Vector Laboratories, Burlingame, Calif.,) product number SK4200, resulting in a brown color in regions containing bound antibodies. Slides were then washed with PBST. Finally, slides were counterstained with Mayer's Hematoxylin for 5 minutes, rinsed and air dried before being mounted with coverslips using aqueous mounting medium (Vector Laboratories, Burlingame, Calif.,) product name: Vectamount. Representative images from immunochemically-treated tissue sections were analyzed.

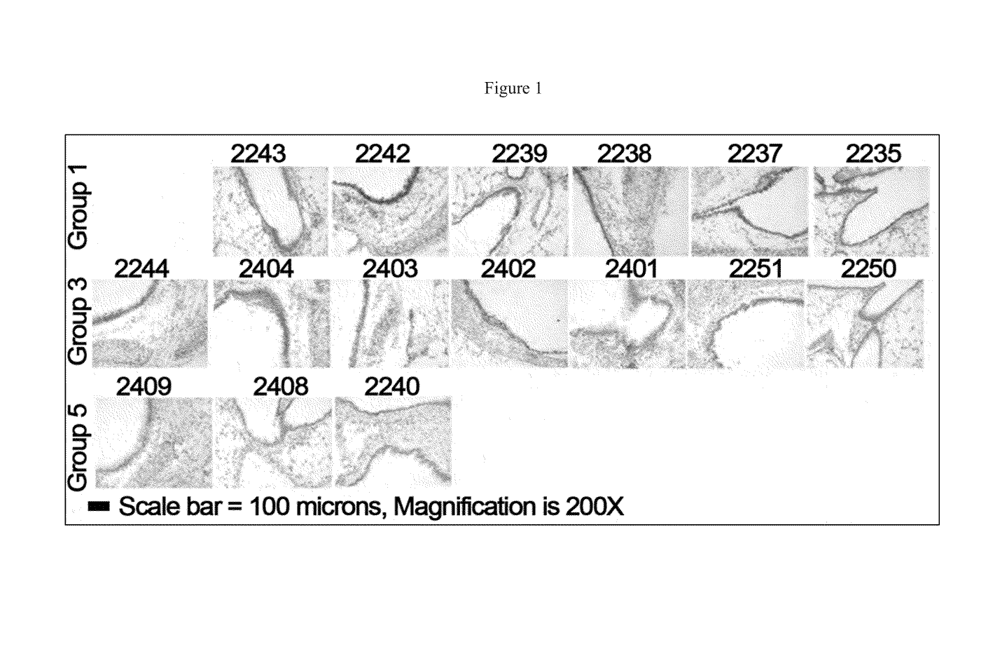

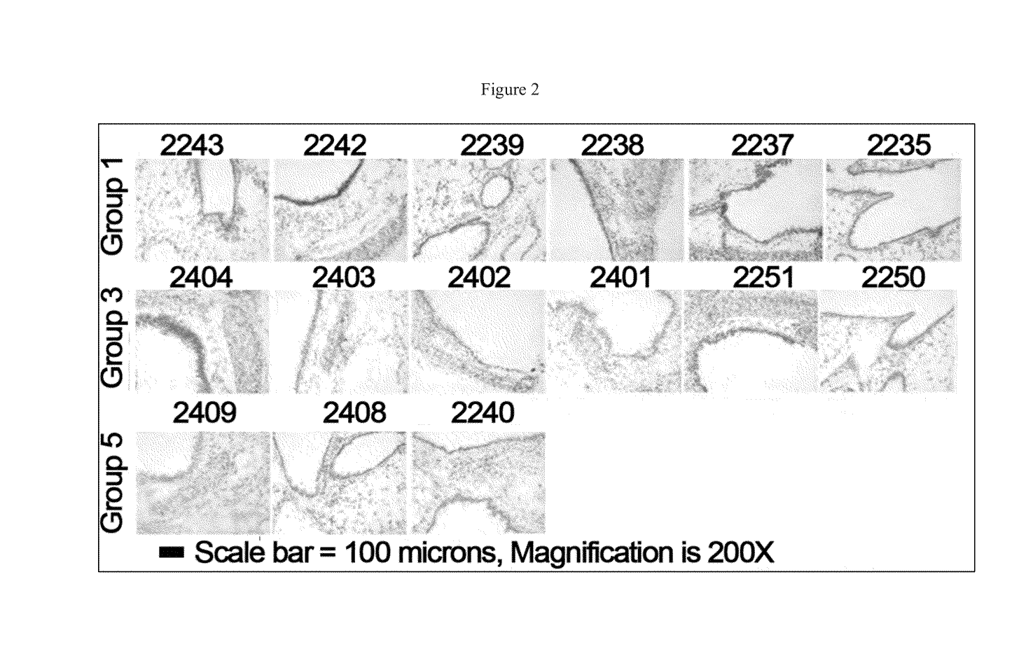

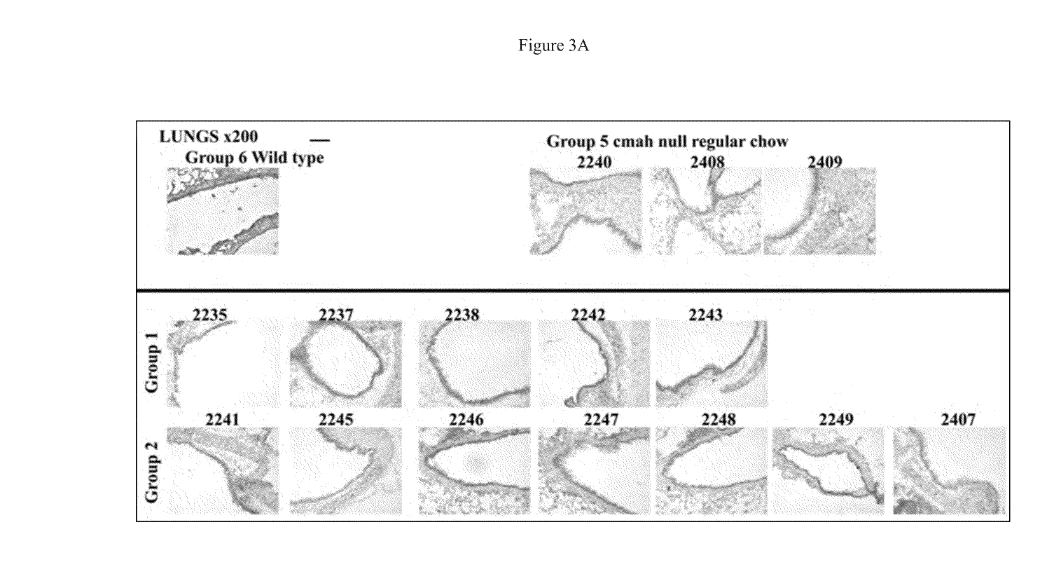

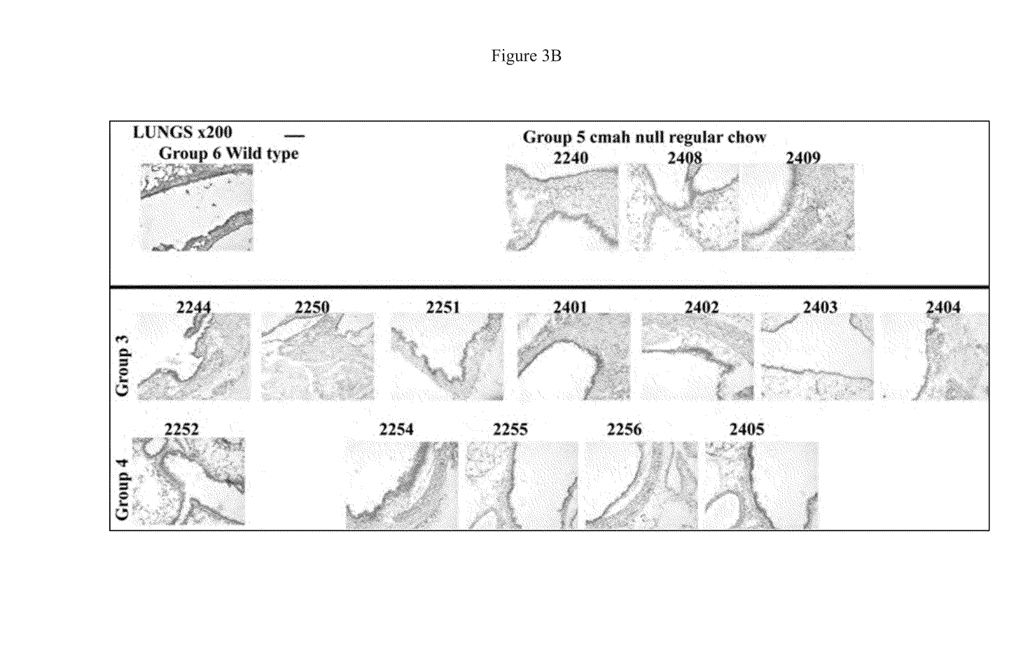

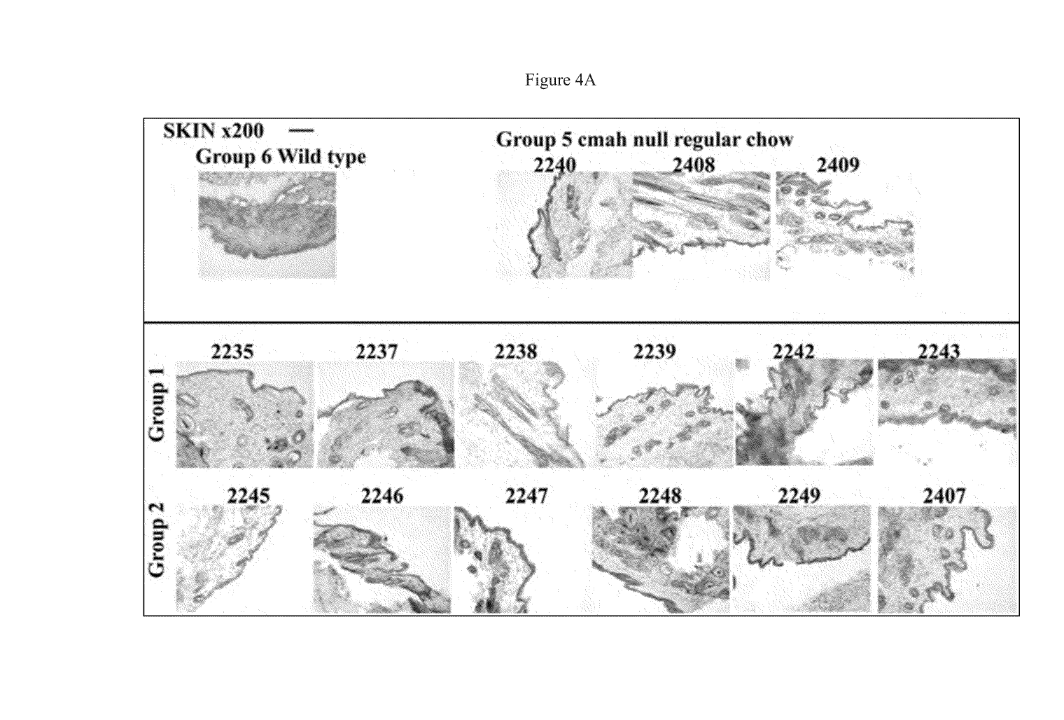

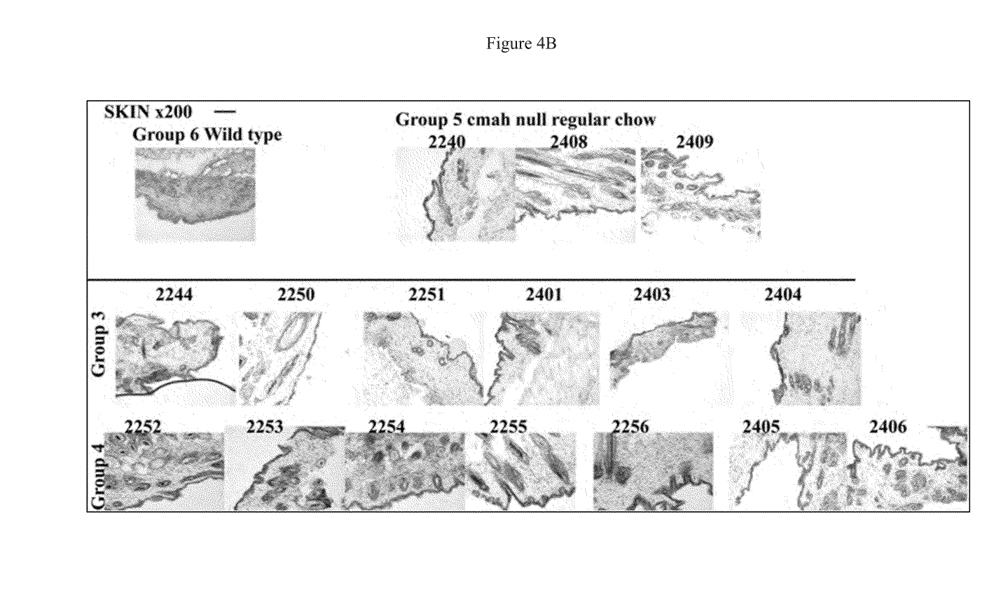

Tracheal images demonstrated a higher level of antibody staining in Group 1 mouse samples [both untreated (see FIG. 1) and periodate treated (see FIG. 2)] as compared to those from Group 3. Group 5 mouse sections did not present detectable antibody staining Antibody staining of Neu5Gc was also detected in lung (FIGS. 3A and 3B) and skin samples (FIG. 4A and FIG. 4B;) however, staining in liver, pancrease and small intestine samples was not convincing due to variability among samples.

### Example 10

### Processed Meat Preparation

Neu5Ac-enriched glycomacropeptide (GMP) is mixed with ground beef. The resulting processed meat product is used to generate beef products for human consumption.

1-17. (canceled)

18. A method of reducing inflammation comprising providing to a patient in need thereof a nutraceutical composition, said nutraceutical composition comprising glycomacropeptide (GMP) and wherein levels of each of interferon γ, interleukin (IL)-5, IL-6, keratinocyte chemoattractant, monocyte chemotactic protein 1, and tumor necrosis factor α are reduced.

19. (canceled)

20. (canceled)

21. A method of evaluating the ability of a nutraceutical composition to modulate inflammation in vivo comprising:
a. administering said nutraceutical composition to a mammal, said nutraceutical composition comprising GMP,
b. obtaining a first sample from the mammal,
c. obtaining a second sample from the mammal, wherein said second sample is obtained from about 1 week to about 12 weeks after obtaining said first sample,
d. determining the level of interferon γ, IL-5, IL-6, keratinocyte chemoattractant, monocyte chemotactic protein 1, and tumor necrosis factor α in said first sample and in said second sample, and
e. comparing the level of interferon γ, IL-5, IL-6, keratinocyte chemoattractant, monocyte chemotactic protein 1, and tumor necrosis factor α determined in said second sample to the level of interferon γ, IL-5, IL-6, keratinocyte chemoattractant, monocyte chemotactic protein 1, and tumor necrosis factor α determined in said first sample.

22. A method of incorporating sialic acid into one or more tissues comprising administering to a patient in need thereof a nutraceutical composition, said nutraceutical composition comprising GMP.

23. The method of claim 22, wherein said one or more tissues include at least one of tracheal, lung, and skin tissue.

24-33. (canceled)

34. The method of claim 22, wherein the level of N-glycolylneuraminic acid (Neu5Gc) is reduced in said one or more tissues.

35. The method of claim 34, wherein said one or more tissues include at least one of tracheal, lung, and skin tissues.

36. The method of claim 21, wherein the level of interferon γ, IL-5, IL-6, keratinocyte chemoattractant, monocyte chemotactic protein 1, and tumor necrosis factor α in said first sample and said second sample are determined by enzyme-linked immunoassay.
